(19) United States
(12) Patent Application Publication
(10) Pub. No.: US 2006/0257858 A1
(43) Pub. Date: Nov. 16, 2006

(54) Methods and compositions for introducing biopolymers into cells

(75) Inventors: Henry Chiou, Encinitas, CA (US); Alfred R. Sundquist, San Diego, CA (US); Weixing Chen, San Diego, CA (US); Paul E. Cizdiel, San Marcos, CA (US)

Correspondence Address: BIOTECHNOLOGY LAW GROUP;C/O PORTFOLIOIP, PO BOX 52050, MINNEAPOLIS, MN, 55402, US

(73) Assignee: INVITROGEN CORPORATION, Carlsbad, CA (US)

(21) Appl. No.: 11/396,287

(22) Filed: Mar. 31, 2006

Related U.S. Application Data
(60) Provisional application No. 60/667,707, filed on Apr. 1, 2005.

(51) Int. Cl. C12Q 1/70; C12Q 1/68

(52) U.S. Cl. 435/5; 435/6; 977/802

(57) ABSTRACT

The invention relates to methods for introducing target biopolymers (e.g., nucleic acid molecules, proteins, etc.) and other compounds (e.g., non-polymeric organic molecules, such as steroids) into cells (e.g., eukaryotic cells). The invention further relates to compositions comprising target biopolymers and other compounds. In certain aspects, the invention relates to the deposition of target biopolymer-containing mixtures onto surfaces optionally followed by contacting these target biopolymer-containing mixtures with cells (e.g., eukaryotic cells). Additionally, when the target biopolymers are nucleic acid molecules, expression of these nucleic acid molecules by cells which are contacted with the target biopolymers may be monitored or detected.

### CROSS-REFERENCE TO RELATED APPLICATION

This patent application claims the benefit of U.S. provisional patent application No. 60/667,707, filed Apr. 1, 2005, entitled “Methods and Compositions for Introducing Biopolymers into Cells,” naming Henry Chiou et al. as inventors and having attorney docket number INV-1006-PV, which is incorporated herein by reference in its entirety.

### FIELD OF THE INVENTION

The invention relates to methods for introducing target biopolymers (e.g., nucleic acid molecules, proteins, etc.) and other compounds (e.g., non-polymeric organic molecules, such as steroids) into cells (e.g., eukaryotic cells). The invention further relates to compositions comprising target biopolymers and other compounds. In certain aspects, the invention relates to the deposition of target biopolymer-containing mixtures onto surfaces optionally followed by contacting these target biopolymer-containing mixtures with cells (e.g., eukaryotic cells). Additionally, when the target biopolymers are nucleic acid molecules, expression of these nucleic acid molecules by cells which are contacted with the target biopolymers may be monitored or detected.

### BACKGROUND

Genome and expressed sequence tag (EST) projects, as examples, have led to the rapid cataloging and cloning of the genes of higher organisms, including humans. One emerging challenge is to determine the functional roles of the genes and to quickly identify gene products with desired properties. The growing collection of gene sequences and cloned cDNAs demands the development of systematic and high-throughput approaches to characterizing the gene products. The uses of DNA microarrays for transcriptional profiling and of yeast two-hybrid arrays for determining protein-protein interactions are recent examples of genomic approaches to the characterization of gene products (Schena, M. et al., Nature, 10:623 (2000)). Comparable strategies for analyzing the function, within mammalian cells, of large sets of genes are only now being developed. Currently, in vivo gene analysis is typically done on a gene-by-gene scale by transfecting cells with a DNA construct that directs the overexpression of the gene product or inhibits its expression or function. The effects on cellular physiology of altering the level of a gene product is then determined using functional assays.

A variety of transfection methods, such as calcium phosphate coprecipitation, electroporation and cationic liposome-mediated transfection (e.g., lipofection) can be used to introduce molecules (e.g., nucleic acid molecules) into cells and are useful, for example, in studying gene regulation and function. Additional methods, particularly high-throughput assays which can be used to screen, for example, large sets of nucleic acid molecules to identify those encoding products with properties of interest save considerable amounts of time and resources.

Transfected cell arrays may be employed to carry out parallel transfections of populations of cells. This may be done by spotting a small amount of a target biopolymer, such as a transfection vector, or other compound on a support and then “seeding” the support with live cells. Under certain circumstances, cells may adhere to the entire surface of the support but only cells that are positioned on the spot (i.e., feature) are exposed to the target biopolymer. When the support contains multiple spots and each spot contains, for example, a unique target biopolymer, then the outcome is essentially an array of cells that have been transfected with different target biopolymers.

To generate overexpression an array, for example, a collection plasmids may be pre-mixed with a transfection reagent, such as Lipofectamine™ 2000, and spotted on a support. As explained below, the inventors have found that in some instances, the addition of other materials to the spotting solution, such as proteins and sucrose, has proven beneficial. Knockdown arrays can be prepared in a similar manner with RNA. This process is generally referred to as reverse transfection and can be carried out planar supports such as 1 inch×3 inch microscope slides or in wells of a microtiter plate.

The preparation of commercially viable transfected cell arrays will depend, in part, on strict control of the arraying process to facilitate the production of reproducible and reliable transfected cell arrays, and on the implementation of measures to control long-term stability of the arrayed transfection agent. Here we describe formulations and methods which may be employed to achieve these ends.

### SUMMARY

The invention relates, in part, to compositions comprising (1) target biopolymers (e.g., nucleic acid molecules, proteins, etc.) and/or and other compounds (e.g., non-polymeric organic compounds, such as steroids) and (2) supports (e.g., solid supports). The invention further relates to methods for using these compositions to introduce target biopolymers and/or other compounds into cells. The invention also relates to methods for introducing target biopolymers and/or other compounds into cells, followed detecting direct or indirect activities of those target biopolymers and/or other compounds upon the cells. Additionally, the invention relates to arrays of cells which contain target biopolymers and/or other compounds.

In particular aspects, the invention relates to compositions comprising a support with a plurality (e.g., two, three, four, five, ten, fifteen, twenty, fifty, seventy, one hundred, three hundred, five hundred, one thousand, two thousand, five thousand, eight thousand, ten thousand, etc.) of nucleic acid molecules which differ in nucleotide sequence localized at different locations on or in the support. In certain specific aspects, the support is a solid planar support such as a glass microscope slide.

In particular aspects, the invention includes compositions comprising supports and two or more features, wherein at least one of the non-control features contains two or more target biopolymers.

In other particular aspects, the invention further includes compositions comprising supports and two or more features, wherein each feature contains one or more (e.g., one, two, three, four, five, six, seven, eight, nine, ten, etc.) carbohydrate.

In additional particular aspects, the invention includes compositions which comprise a support (e.g., a solid support) with two or more features, wherein each feature comprises viral particles or viruses which contain nucleic acid molecules and the nucleic acid molecules in different features differ in nucleotide sequence. In a more specific aspect, the viral particles and/or viruses may be co-localized in the features on the support with one or more deposition agents.

Depositing agents used in various aspects of the invention may contain at least one (e.g., one, two, three, four, five, six, seven, etc.) protein (e.g., a protein other than a protein normally associated with the viral particles and/or viruses of the features). Proteins present in deposition agents include cationic proteins (e.g., protamines). Exemplary proteins which may be present is deposition agents include an albumin (e.g., bovine serum albumin), a histone (e.g., an H1 or an H4 histone), or a fibronectin (e.g., a bovine fibronectin).

Depositing agents used in various aspects of the invention may also contain at least one monosaccharide (e.g., glucose, ribose, ribulose, etc.), disaccharide (e.g., sucrose, maltose, galactose, etc.), trisaccharide, or polysaccharide. Similarly, depositing agents may contain at least one complex carbohydrate, such as a gum (e.g., guar gum, gum arabic, xanthan gum, tragacanth, locust bean gum, etc.), a pectin, or a carrageenan (e.g., kappa-carrageenan, lambda-carrageenan, iota-carrageenan, etc.). In particular aspects, deposition agents may contain at least one hydrocolloid gum.

In further aspects of the invention, viral particles and/or viruses of at least one feature are co-localized on support with one or more transfection reagent.

The number of features on the support of compositions of the invention will vary with the particular application. For example, if the features contain expression vectors designed to produce kinases, the number of features will typically be limited to the number of kinases which one desires to test in a cell type which is contacted with the support and any control features present (e.g., features which contain an expression vector designed to allow for the expression of GFP, beta-lactamase, or other suitable reporter). Often the number of features present will be in a range of from about 2 to about 10,000, from about 10 to about 10,000, from about 100 to about 10,000, from about 500 to about 10,000, from about 1,000 to about 10,000, from about 2,000 to about 10,000, from about 50 to about 1,000, from about 500 to about 2,000, from about 1,000 to about 5,000, from about 1,000 to about 2,000, from about 2,000 to about 4,000, from about 1,500 to about 5,000, from about 1,500 to about 4,000, from about 30 to about 500, from about 40 to about 1,000, or from about 100 to about 1,000.

Of course, the number of features present will also depend upon factors such as the size and shape of the support and the feature density. The feature density will be limited by technical factors such as (1) the amount of target biopolymer present in each feature, (2) the amount of target biopolymer which must be introduced into cells in order to detect an effect, and (3) the number of cells which must be contacted with each feature in order to generate representative data of the cellular population. Regarding the last item, in many instances, in order to generate reliable data, target biopolymers will need to be introduced into a particular number of cells (e.g., at least 50 or 100 cells). As an example, in many instances, if the feature is of such a size that a single cell contains it, the data derived from that feature will result from the transfection of that single cell. In many instance, this will not lead to consistent data.

The number of cells which must be contacted with each feature in order to generate reliable data will vary with the particular experiment and such factors as (1) the amount of biopolymer in each feature, (2) the target biopolymer itself, (3) the transfection efficient, (4) the cells which are contacted with the feature, and (5) other conditions. Typically, the average number of cells which will be contacted with each feature will be greater than 50 (e.g., at least 55, 75, 100, 150, 200, 300, etc.). Often the average number of cells contacted with each feature will be in the range of from about 50 to about 2,000, from about 50 to about 1,000, from about 50 to about 500, from about 50 to about 200, from about 100 to about 2,000, from about 100 to about 1,000, from about 100 to about 500, from about 200 to about 2,000, from about 200 to about 1,000, or from about 200 to about 500.

In particular embodiments, the density of features on supports of the invention will be in the range of from about 2 to about 100 features per cm2, from about 5 to about 300 features per cm2, from about 10 to about 100 features per cm2, from about 10 to about 2,000 features per cm2, from about 100 to about 2,000 features per cm2, from about 200 to about 2,000 features per cm2, from about 400 to about 2,000 features per cm2, or from about 800 to about 2,000 features per cm2.

Supports of the invention will often be solid supports or semi-solid supports. Exemplary solid support include glass plates such as glass microscope slides. Exemplary semi-solid support include agarose surfaces. In many instances, a semi-solid support will be positioned on a rigid surface.

Target biopolymers which may be used in the invention include polymeric molecules such as nucleic acid molecules, proteins, or polysaccharides. In particular embodiments, supports of the invention may contain at least one feature with nucleic acid molecules which are either RNA molecules or DNA molecules, or a combination of RNA molecules and DNA molecules.

When the target biopolymer in at least one feature is a nucleic acid molecule, this nucleic acid molecule may encode a double-stranded RNA and/or a protein. This double-stranded RNA may be capable of, for example, knocking down gene expression by RNA interference. Additionally, the nucleic acid molecule may encode a functional RNA such as a ribozyme, transfer RNA (tRNA), etc.

When the nucleic acid molecule encodes a tRNA, for example, cells can be transfected with the nucleic acid molecule and then screened for those cells which require, for example, a suppressor function to generate a particular phenotype. As a specific example, a nucleic acid molecule (e.g., DNA) which encodes a suppressor tRNA may be located within a feature along with an antibiotic (e.g., tetracycline). Further, different support features may contain different antibiotics. Cells which contain a reporter gene located down stream from a tetracycline responsive regulatory element (see, e.g., Invitrogen Corporation's T-Rex™ System, cat. nos. K1030-02, V1033-20, and K1020-01). The reporter gene contains a suppressible stop codon. Thus, reporter gene activity can be detected when cells are contacted with a feature which contains both DNA which encode the suppressor tRNA and tetracycline. In this way, drug screens may be designed using methods of the invention.

Any number of variations of the above are possible. For instance, features may contain a mixture comprising potential activators and potential inhibitors of gene expression. Arrays containing such features may be used to screen for cells which respond to certain activators and/or inhibitors. As an example, a feature may contain a protein or other molecule which is capable of activating gene expression and a molecule which is to be screened for activity related inhibition of gene expression. In a more specific example, the activator may be tetracycline, as described above, and the potential inhibitor may be an RNAi molecule. When different features of an array contain different RNAi molecules, these features can be used to quantify the ability of the RNAi molecules to knock down gene expression. For instance, the target gene may be expressed in cells contacted with the features as a fusion protein, wherein the fusion protein contains a region which is potentially targeted by the RNAi molecules of the individual feature and a region with reporter activity (e.g., beta-lactamase activity, green fluorescent protein (GFP), yellow fluorescent protein (YFP), cyanine fluorescent protein (CFP), etc.).

In embodiments described herein, the activator of gene expression may vary upon the specific system used. One example of an activator is TNF-alpha, which can be used to activate gene expression in cells which have the appropriate responsive element. One example of such cells are the CellSensor™ NFB-bla ME-180 cell line (Invitrogen. Corp., cat. no. K 1171) which contains a stably integrated beta-lactamase reporter gene under control of a Nuclear Factor Kappa B (NFB) response element.

The invention thus includes methods for screening molecules, such as drugs and drug candidates, for cellular effects. These effects may be mediated by, for example, genes normally present within the cells genome or introduced by recombinant nucleic acid techniques (e.g., the introduction of a reporter gene by homologous recombination).

When a target biopolymer of at least one support feature is an RNA molecule, this RNA molecule may also encode another RNA molecule or a protein. Additionally, the RNA molecule itself may be functional without undergoing transcription or translation. For example, the RNA molecule may be a ribozyme or double-stranded RNA molecule which is capable of mediating RNA interference.

Carbohydrates which may be used in the practice of the invention include monosaccharides, disaccharides, trisaccharides, and polysaccharides (i.e., a carbohydrate with four or more monosaccharide monomers). Further, these carbohydrates may be complex carbohydrates, such as gums, pectins, or carrageenans. In particular embodiments, the gum may be a hydrocolloid gum (e.g., guar gum, xanthan gum, locust bean gum, etc.). Deposition agents used herein may contain the carbohydrates referred to above, as well as other carbohydrates.

Features may contain, in addition to one or more target biopolymer, at least one transfection reagent. In particular embodiments, features will contain one or more target biopolymer, one or more transfection reagent, and one or more carbohydrate.

The invention further includes methods for introducing target biopolymers and/or other compounds into cells. In particular embodiments, the methods comprise contacting the cell with supports which contain two or more (e.g., two, three, four, five, six, seven, eight, nine, ten, fifteen, twenty, thirty, forty, eighty, one hundred, five hundred, one thousand, two thousand, etc.) features, wherein the cells are contacted with individual locations on the supports which contain either (a) target biopolymers and/or other compounds and depositing agents containing at least one carbohydrate (e.g., at least one gum) or (b) viral particles and/or viruses which contain the target biopolymers and/or other compounds.

As above for compositions of the invention, when features contain viral particles and/or viruses, these features may contain one or more transfection reagent or other suitable reagents or compounds.

Also as above for compositions of the invention, target biopolymer of the features may be nucleic acid molecules, such as expression vectors. Further, these expression vectors may encode molecules such as proteins or expression double-stranded RNAs. Double-stranded RNA molecules which are either expressed from nucleic acid molecules contained within features or that are themselves present within features may vary greatly in size. Further the size of the double-stranded RNA molecules will often depend on the cell type contacted with the feature. As an example, animal cells such as those of C. elegans and Drosophila melanogaster do not generally undergo apoptosis when contacted with double-stranded RNA molecules greater than about 30 nucleotides in length (i.e., 30 nucleotides of double-stranded region) while mammalian cells typically do undergo apoptosis when exposed to such double-stranded RNA molecules. Thus, the design of the particular experiment will often determine the size of double-stranded RNA molecules contained within or encoded by nucleic acid molecules of the features.

In many instances, the double-stranded region of double-stranded RNA molecules contained within or encoded by nucleic acid molecules of the features will be within the following ranges: from about 20 to about 30 nucleotides, from about 20 to about 40 nucleotides, from about 20 to about 50 nucleotides, from about 20 to about 100 nucleotides, from about 22 to about 30 nucleotides, from about 22 to about 40 nucleotides, from about 20 to about 28 nucleotides, from about 22 to about 28 nucleotides, from about 25 to about 30 nucleotides, from about 25 to about 28 nucleotides, from about 30 to about 100 nucleotides, from about 30 to about 200 nucleotides, from about 30 to about 1,000 nucleotides, from about 30 to about 2,000 nucleotides, from about 50 to about 100 nucleotides, from about 50 to about 1,000 nucleotides, or from about 50 to about 2,000 nucleotides. The ranges above refer to the number of nucleotides present in double-stranded regions. Thus, these ranges do not reflect the total length of the RNA molecules themselves. As an example, a blunt ended double-stranded RNA molecule formed from a single transcript of 50 nucleotides in total length with a 6 nucleotide loop, will have a double-stranded region of 23 nucleotides.

The invention further includes methods for making compositions comprising supports with two or more features. In one aspect, these methods include those which comprise depositing viral particles and/or viruses on supports to produce the two or more features on each support. In another aspect, these methods include those which comprise depositing mixtures of one or more target biopolymer and a depositing agent containing one or more carbohydrate (e.g., one or more gum) on supports to produce the two or more features.

The invention additionally includes arrays of target biopolymers comprising two or more dried spots on supports, wherein at least two of the dried spots on each support comprise at least one target biopolymer and a depositing agent containing one or more carbohydrate (e.g., one or more gum). As above for other compositions of the invention, one or more feature of such arrays may further comprise one or more transfection reagent.

The invention further include methods of screening for at least one activity of a target biopolymer. In particular embodiments, such methods comprise (a) contacting a population of cells with an array comprising two or more features which each contain different biopolymers and (b) evaluating the contacted cells to detect a direct or an indirect activity associated with the introduction of at least one of the target biopolymers into the contacted cells. As noted elsewhere herein, molecules other than target biopolymers (e.g., drugs, drug candidates, etc.) may be substituted for the target biopolymers in this and other embodiments of the invention. As an example, if one desired to compare the effect that non-steroidal anti-inflammatory drugs (NSAIDs) have on human cells, then one could prepare an array of such drugs for use in methods of the invention. Along these lines, there are currently approximately 25 NSAIDs (e.g., Bextra™, Vioxx™, Celebrex™, Daypro™, naproxyn, etc.) which either are currently have been sold or have recently been sold for the treatment of inflammatory afflictions. Thus, the invention includes methods for screening drugs and drug candidates for effects these compounds have on cells.

In some instances, molecules which are contacted with cells using methods and compositions of the invention need not enter the cells to mediate their effect. In particular, these molecules may mediate their effect by transmembrane signal transduction. Thus, the invention includes methods and compositions for inducing cellular effects, wherein the effect is mediated through transmembrane signal transduction.

The invention further includes kits which contain components referred to herein. In particular embodiments, the invention includes kits which comprise one or more of the following compositions: (a) a support with two or more features, wherein each feature comprises viral particles and/or viruses which contain nucleic acid molecules and the nucleic acid molecules in different features differ in nucleotide sequence; (b) a support with two or more features, wherein each feature contains one or more carbohydrate; or (c) an array of target biopolymers comprising two or more dried spots on a solid support, wherein at least two of the dried spots comprise at least one target biopolymer and a depositing agent containing one or more gum.

Kits of the invention may further comprise one or more of the following components: (a) one or more cell line, (b) one or more buffer, (c) one or more enzyme; (d) one or more transfection reagent; (e) one or more culture medium or culture medium component; and (f) one or more set of instructions for using one or more kit component.

The invention further includes commercial methods and products designed, for example, to attract customers to purchase products which contain methods and compositions of the invention. In particular aspects, the invention includes methods for supplying products to customers. These methods include those which comprise: (a) taking an order for the particular product and (b) supplying the product to the customer. In many instances, the product with be a composition described herein such as a kit. Methods of the invention further include activities related to accounting functions associated with the sale of products. As an example, methods of the invention include those wherein a bill is provided to the customer/purchaser of the product. Such bills may be shipped along with the product or may be sent separately from the product.

The invention further includes methods for advertising products. In particular aspects, these method comprise: (a) preparing an advertisement for the product and (b) publicly disclosing the advertisement. In many instances, the product with be a composition described herein, such as a kit described above. Exemplary advertisements of the invention include: (a) magazine advertisements, (b) flyers or brochures designed for mailing or otherwise giving to customers or potential customers, (c) web pages or web accessible documents, and (d) posters designed to be presented at scientific meeting or other public functions.

### BRIEF DESCRIPTION OF THE DRAWINGS

FIG. 1 shows a format for an exemplary composition of the invention. The support in this instances is a glass microscope slide (1) with 100 features represented by ninety-six filled circles (2) and four unfilled circles (3, 4). The filled circles represent features with various non-control target biopolymers. The unfilled circles represent features with control target biopolymers. Some of the unfilled circles (3) contain control target biopolymers, such as an expression vectors which encodes a reporter, or other control compound. Other unfilled circles (4) contain no target biopolymer or other compound to be introduced into contacted cells. Thus, these unfilled circles (4) are blanks.

FIGS. 2A-2E show vector formats which may be used in methods of the invention. FIG. 2A shows a vector format which may be used to produce, for example, single transcripts (e.g., mRNAs, tRNAs, ribozymes, double-stranded RNA molecules, etc.) FIGS. 2B-2E show vector formats which may be used to produce, for example, double-stranded RNA molecules. “P” refers to a promoter. “TS” refers to a transcribed sequence. “TS-F” refers to a transcribed sequence which is in a forward orientation. “TS-R” refers to a transcribed sequence which is in a reverse orientation. Forward and reverse here refer to formats which allow for the synthesis of complementary RNA strands which, under suitable conditions, are capable of hybridizing to each other. “L” in FIG. 2C refers to a linker which connects the forward and reverse transcribed sequences.

FIG. 3 shows bright field images of transfection spots, also referred to herein as “features”, prepared with various guar gum concentrations on an amine modified glass microscope slide (GAPS II, Corning Inc., Acton, Mass., cat. no 40003). The features were prepared from solutions composed of 30 μg/ml GFP plasmid, 180 μg/ml Lipofectamine™ 2000, 100 mM sucrose and 0.5 mg/ml human fibronectin. The features were generated by hand by dispensing 0.15 μl spots onto the slide with an electronic Rainin pipetor (model EDP3-LTS 10).

FIG. 4 shows GFP transfection of GripTite™ cells (Invitrogen Corp., Carlsbad, Calif., cat. no. R795-07) on spots (i.e., features) prepared with various concentrations of guar and xanthan gum. Images were acquired by fluorescence microscopy (40×). The array was prepared by manually spotting a DNA/lipid complex (30 μg/ml GFP plasmid, 180 μg/ml Lipofectamine™ 2000, 100 mM sucrose and 0.5 mg/ml human fibronectin) on a GAPS II slide. The average diameter of the dried spot was 1.2 millimeters, and spot spacing was about 2 millimeters center-to-center.

FIG. 5 shows GFP transfection of GripTite™ cells on spots prepared with various proteins added to transfection complex over a range of concentrations. Images were acquired by fluorescence microscopy (40×).

FIG. 6 shows transinfection of GripTite™ cells on spots of lentivirus containing GFP. Lentivirus arrays were prepared with guar gum and stored at 4° C. under desiccation. Images were acquired by fluorescence microscopy (40×). In brief, the lentivirus used was a virus that contained an open reading frame which encodes GFP. After introduction into GripTite™ cells, GFP expression was observed in the cells. For making lentivirus spots on GAPS II slides, lentivirus samples were added into DMEM medium containing sucrose and guar, 0.5 μl of the mixture then was spotted on the GAPS II slides. The slides were stored at 4° C. under desiccation up to 8 weeks until reverse transfection was performed.

FIG. 7 Lipofectamine™ 2000 reverse transfection of Emerald GFP vector into 293 GripTite™ cells with various matrixes on a GAPS II slide. 0.15 μl lipid/DNA mixture/per spot. The mixtures contain 100 mM sucrose and fibronectin (bovine) or polybrene at concentrations as indicated as above. The final DNA concentration in the mixture is 30 ng/μl. Lipid is Lipofectamine™ 2000. Lipid/DNA ratio: 6/1. The lipid/DNA reaction occurs in water for 1 hour. The printed slide was dried for 3 hours and then covered by cells. Cell density: 6.2×105/ml×8 ml in a quadriPERM dish. The photos were taken 20 hours after reverse transfection.

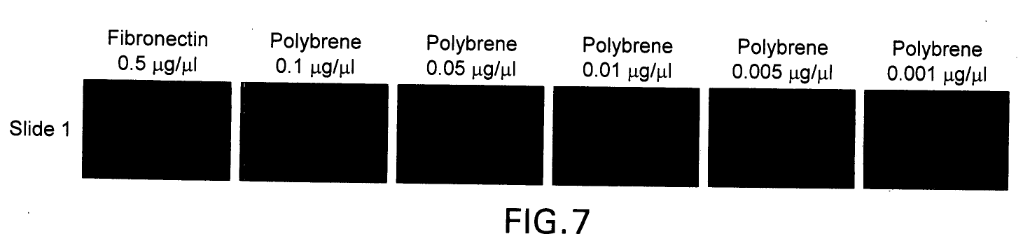

FIG. 8 shows data similar to that of FIG. 7 except that the mixtures contain 100 mM sucrose and fibronectin (bovine) or fetal bovine serum (FBS).

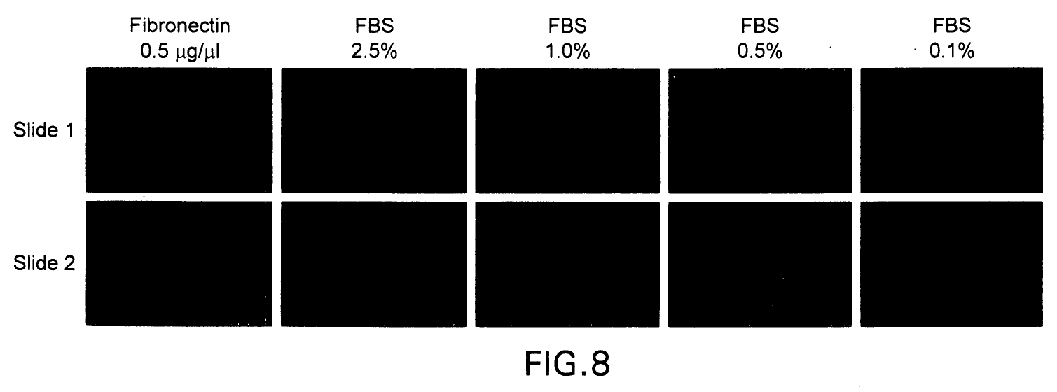

FIG. 9 shows data similar to that of FIGS. 7 and 8 except that the mixtures contain 100 mM sucrose and fibronectin (bovine) or glycerol.

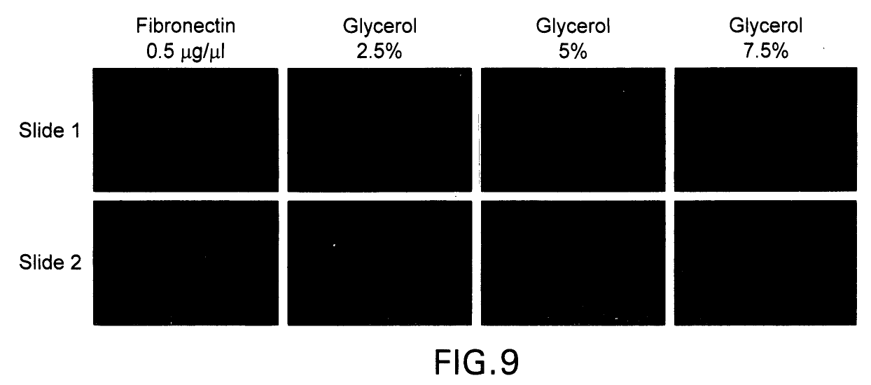

FIG. 10 outlines a transfected cell array-based approach to expression cloning with cDNA libraries. Overall, the technique shown is similar to classical sib-selection except that the library is first plated and individual clones are randomly picked, archived and pooled. Nucleic acid molecules from the pooled clones (sub-libraries) are then complexed with appropriate deposition reagent and/or other compounds (e.g., a transfection reagent) that facilitate transfection, and spotted on an array. The array itself, containing features of sub-libraries may be a commercial product which can be purchased and employed in genetic screening assays. “Hits” are phenotypic responses specific to assay which are displayed by cells transfected nucleic acid molecules of particular feature(s). The identity of the clones in that feature (like all features), may be unknown. By knowing information such as the feature coordinates, ID numbers for the clones comprising the sub-library present in that feature can be accessed through the internet or a database file supplied with each array. Each clone can then be ordered individually from the supplier by referencing, for example, the clone ID number. Finally, the user will often need to repeat the genetic screening assay on each clone independently, to determine which particular clone was responsible for eliciting the desired phenotypic response. That clone may then be sequenced and characterized.

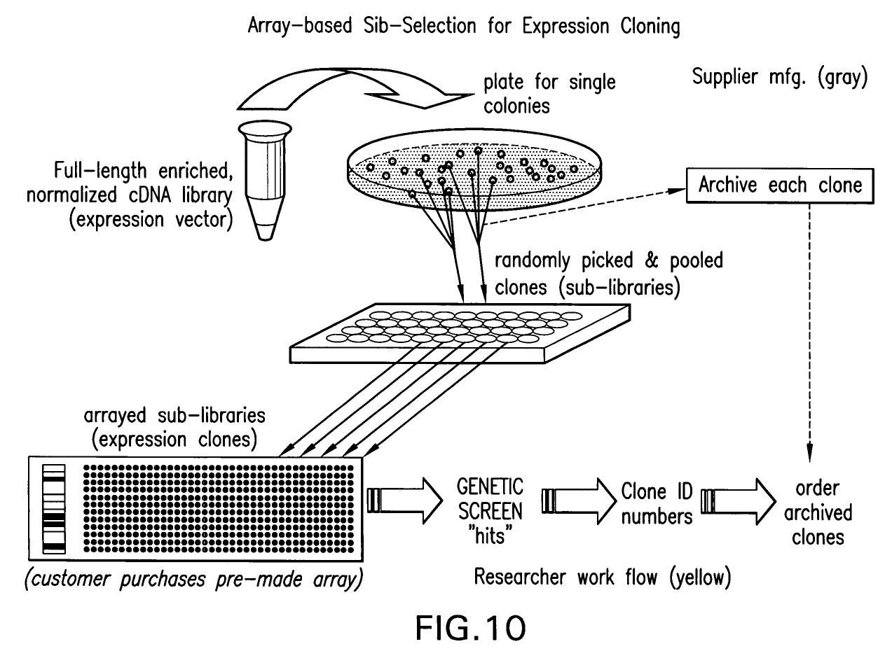

FIG. 11 show Lipofectamine™ 2000 reverse transfection of an Emerald GFP vector into 293 GripTite™ cells with various matrixes on a GAPS II slide. The spots were hand-printed on the GAPS II slide. 0.15 μl of lipid/DNA mixture was applied per spot. The mixtures contain 100 mM sucrose and various matrix proteins as indicated in figure. The final DNA concentration in the mixture was 30 ng/μl and the lipid/DNA ratio was 6:1. The lipid/DNA reaction occurred in water. The printed slide was dried in a sealed aluminum bag with desiccation at room temperature for 3 days. The slide was then covered by 293 GripTite™ cells in a quadriPERM 4 chamber dish (one slide per chamber). Cell density: 6.5×105/ml×8.5 ml per chamber of a quadriPERM 4 chamber dish. The photos were taken 48 hours after reverse transfection.

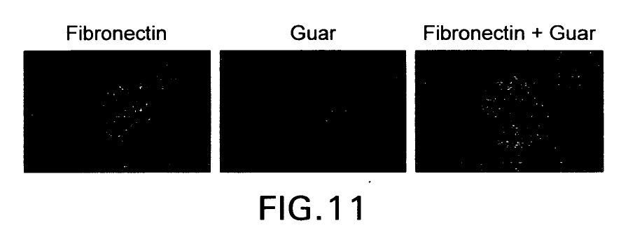

FIG. 12 provides a schematic representation of a system for providing a product to a party such as a customer/purchaser.

FIG. 13 provides a schematic representation of a system for advising a party as to the availability of a product.

The patent or application file contains at least one drawing executed in color. Copies of this patent or patent application publication with color drawings, will be provided by the Office upon request and payment of the necessary fee.

### DETAILED DESCRIPTION

I. Definitions

Before further description of the invention, certain terms employed in the specification, examples and appended claims are, for convenience, collected here.

As used herein, the term “deposition agent” means a composition which may be (1) mixed with one or more target biopolymers (e.g., nucleic acid molecules, protein molecules, polysaccharide molecules, etc.) or other compounds that one seeks to introduce into cells and (2) then applied to a support. Typically, a deposition agent will have one or more of the following characteristics: (1) inexpensive; (2) capable of stabilizing the mixture to be spotted prior to spotting; (3) allow for easier spotting of the solutions on supports; (4) allow for drying of the spots on supports; (5) enhance stability of the dried spots; (6) allow for uptake of biological polymers by cells which are contacted with supports that contain the biological polymers; and (7) enhance the exposure of cells contacted with supports to transfection reagents.

The term “deposition agent” does not include transfection reagents. However, in many instances, deposition agents will be mixed with transfection reagents.

As used herein, the term “transfection” refers to the introduction of one or more exogenous compound (e.g., biological polymers) into cells.

As used herein, the term “complex carbohydrate” refers to a polymeric carbohydrate of at least ten monomeric sugars. Thus, the term complex carbohydrate includes pectins, gums and starches. Often the monomeric sugars of a complex carbohydrate will be pentoses and hexoses. The term complex carbohydrate does not include nucleic acids.

As used herein, the term “disaccharide” refers to a carbohydrate composed of two monomeric sugars. Thus, the disaccharide includes sucrose, maltose, and galactose.

As used herein, the term “gum” refers to plant-derived (isolated from exudates or extracts) polysaccharides that are members of a category of hydrophilic compounds called hydrocolloids. These compounds typically have elastoviscous properties when mixed with water. In many instances, a gum will harden upon drying.

As used herein, the term “transfection reagent” refers to chemical compositions which enhances the ability of nucleic acid molecules to enter cells. A considerable number of transfection reagents are known in the art and include cationic and anionic lipids and polyamines. A number of exemplary transfection reagents are referred to elsewhere herein. Additional examples of transfection reagents are described in U.S. Patent Publication Nos. 2004/0077582 and 2004/0077888, the entire disclosures of which are incorporated herein by reference.

As used herein, the term “nucleic acid molecules” refers to polynucleotides such as deoxyribonucleic acid (DNA), and, where appropriate, ribonucleic acid (RNA). Also included in this term are chemically modified nucleic acid molecules such as Stealth™ RNAi molecules (available from Invitrogen Corp., Carlsbad, Calif.).

As used herein, the term “cDNA” refers to DNA which has been generated by reverse transcription of mRNA and from which introns have been removed. As used herein, the term “heterologous nucleic acid” refer to nucleic acid (e.g., DNA or RNA) that does not occur naturally as part of the genome in which it is present or does naturally occur as part of the genome but has been introduced as part of another nucleic acid molecule which is not naturally present in the cell into which it is introduced. Thus, a plasmid cloning vector would be considered a heterologous nucleic acid even if it contains an insert which has a nucleic acid segment with a nucleotide sequence which is identical to the nucleotide sequence of a segment of the cell's chromosome.

As used herein, the term “viral particle” refer to nucleic acid and associated viral coat proteins. Viral particles will often be composed of (1) one or more nucleic acid molecules each of which contains at least one packaging signal and (2) at least one viral protein or a modified form thereof. Generally, the packaging signal will, in essence, mark the nucleic acid molecule(s) for association with (e.g., binding to) the viral protein(s). In many instances, the nucleic acid molecule(s) will be contained within 3-dimensional structure composed of the viral protein(s), as well as, optionally other components (e.g., cellular proteins, cellular lipids, etc.). Exemplary viral particles are those formed by the use of Invitrogen Corporation's Virapower™ Lentiviral and Adenoviral cloning systems (see, e.g., cat. nos. K4930-00, K4940-00, K4950-00 and K4960-00). In some instances, nucleic acid molecules which are intended to be introduced into cells will be attached to the outside of viral particles. In such instances, the viral particles may or may not contain nucleic acids. Thus, the viral particles may be “empty”.

As used herein, the term “target biopolymer” refer to polymeric organic molecules. In most cases, target biopolymers will be transfected into the host cells (i.e., cells into which the biopolymers are introduced or contacted with). In many instances, target biopolymers will be components of transfection arrays. Often, target biopolymers will have the ability to confer changes in the phenotype of the host cells. The target biopolymer can be, for example, a nucleic acid molecule (e.g., a heterologous nucleic acid molecule) which encodes a protein or RNA other than messenger RNA (e.g., anti-sense RNA, ribozyme, or double-stranded RNA). The target biopolymer can also be or a protein or a carbohydrate. Examples of different target biopolymers include expression vectors which differ only in the nucleotide sequence of nucleic acid segments which are operably linked to an expression regulatory sequence (e.g., a promoter).

As used herein, the term “feature” refers to an area of a support (e.g., a solid support) which is relatively homogeneous with respect to the presence of target biopolymer or other molecule(s) which one desires to introduce into or contact with host cells. This does not mean that the concentration of the target biopolymer or other molecule must be identical throughout the feature but that the same target biopolymer(s) or other molecule(s) are present. One feature differs from another feature if the target biopolymer or other molecule(s) of these features are different (e.g., have a different nucleotide sequence, a different amino acid sequence, a different carbohydrate monomer compositions or sequence, etc.). Typically, features will be in the form of spots on a support (e.g., a transfection array).

As used herein, the term “vector” refers to a nucleic acid molecule capable of being transporting into and/or maintained within a cell. Many vectors are capable of autonomous replication in cells and have selection markers. Plasmids are examples of a type of vector.

As used herein, the terms “operatively linked” and “operably connected” refer to the functional relationship of a nucleic acid segment with regulatory and effecter nucleotide sequences, such as promoters, enhancers, transcriptional and translational start and stop sites, and other signal sequences. For example, operative linkage of a nucleic acid segment to a promoter refers to the physical and functional relationship between the nucleic acid segment and the promoter such that the transcription of such the nucleic acid segment is initiated from the promoter by an RNA polymerase that specifically recognizes, binds to, and transcribes the nucleic acid segment.

As used herein, the term “expression” refers to any number of steps comprising the process by which nucleic acids are transcribed into RNA, and, optionally, translated into peptides, polypeptides, or proteins. If the nucleic acid is derived from genomic DNA, expression may, if an appropriate eukaryotic host cell or organism is selected, include splicing of the RNA.

As used herein, the term “recombinant cells” includes any cells that have been modified by the introduction of heterologous nucleic acid. Control cells include cells that are substantially identical to the recombinant cells, but do not express one or more of the products encoded by the heterologous nucleic acid.

The terms “protein,” “polypeptide,” and “peptide” are used interchangeably herein.

As used herein, the term “cell surface receptor” refers to molecules which are typically located on the surface of cells, interact with the extracellular environment, and transmit or transduce the information regarding the environment intracellularly in a manner that may modulate intracellular second messenger activities or transcription of specific promoters. Often, cell surface receptor mediated signal transduction results in transcription of specific genes. The invention includes methods and compositions which allow for the stimulation of cell surface receptors to generate a cellular response.

As used herein, the term “extracellular signals” refers to a molecule or a change in the environment that is transduced intracellularly via cell surface proteins that interact, directly or indirectly, with the signal. An extracellular signal or effecter molecule includes any compound or substance that in some manner alters the activity of a cell surface protein. Examples of such signals include, but are not limited to, molecules such as acetylcholine, growth factors and hormones, lipids, sugars and nucleotides that bind to cell surface and/or intracellular receptors and ion channels and modulate the activity of such receptors and channels. The term also includes as yet unidentified substances that modulate the activity of a cellular receptor, and thereby influence intracellular functions. Such extracellular signals are potential pharmacological agents that may be used to treat specific diseases by modulating the activity of specific cell surface receptors. the invention includes features which contain and/or mediate extracellular signals.

As used herein, the term “reporter vector” refers to a nucleic acid that includes a “reporter gene” operatively linked to at least one transcriptional regulatory sequence (e.g., a promoter). Transcription of the reporter gene is controlled by these sequences to which they are linked. Exemplary reporter genes include green fluorescent protein and beta-lactamase.

“Signal transduction” is the processing of physical or chemical signals from the cellular environment through the cell membrane, and may occur through one or more of several mechanisms, such as activation/inactivation of enzymes (such as proteases, or other enzymes which may alter phosphorylation patterns or other post-translational modifications), activation of ion channels or intracellular ion stores, effecter enzyme activation via guanine nucleotide binding protein intermediates, formation of inositol phosphate, activation or inactivation of adenylyl cyclase, direct activation (or inhibition) of a transcriptional factor and/or activation.

II. Introduction

The invention relates to a wide variety of compositions which comprise target biopolymers (e.g., nucleic acid molecules) associated with supports (e.g., solid supports). Such compositions may be used in any number of ways. One of these ways is for high-throughput, functional screening of target biopolymers which confer particular characteristics upon cells. These characteristics which may be conferred upon a cell may be mediated either directly or as the result of the production of an expression product.

Examples of target biopolymers/support compositions which may be used to screen for the ability of the nucleic acid molecules to confer particular characteristics upon cells indirectly are described, for example, in Sabatini et al., U.S. Pat. No. 6,544,790, the entire disclosure of which is incorporated herein by reference. Compositions described in this patent comprise expression vectors and solid supports. Thus, when cells are overlayed onto these supports, the nucleic acid molecules are taken up by the cells and, under certain circumstances, the production of expression products (e.g., green fluorescent protein) can be detected.

Examples of nucleic acid molecules/support compositions which may be used to screen for properties of the nucleic acid molecules to confer particular characteristics upon cells directly are described, for example, in Sabatini, U.S. Patent Publication No. 2003/0228601, the entire disclosure of which is incorporated herein by reference. Example of compositions which may be used in this manner are supports which comprise double-stranded RNA molecules. Thus, when cells are overlayed onto these supports, the RNA molecules may be taken up by the cells and, under certain circumstances, RNAi mediated knock-down of gene expression can be detected. These RNA molecules are said to directly confer characteristics upon cells because they are directly involved in the RNAi mediated knock-down process.

Thus, arrays of the invention may be used, for example, to analyze the function in cells of many genes in parallel. In particular embodiments, cells are cultured on a glass slide printed in defined locations with solutions containing different target biopolymers (i.e., features). Cells growing on the features take up the target biopolymers, creating spots of localized transfection within a lawn of non-transfected cells. As an example, by printing sets of complementary DNAs (cDNAs) cloned in expression vectors, arrays which comprise groups of live cells that express a defined cDNA at each location can be made. The invention includes such cell arrays, as well as methods for producing such arrays. Transfected cell arrays can be of broad utility for the high-throughput expression cloning of genes, particularly in areas such as signal transduction and drug discovery.

The invention also relates, in part, to a method of introducing defined target biopolymers (e.g., DNAs) into cells at specific discrete, defined locations on a surface by reverse transfection. That is, methods of the invention can be used to make use of nucleic acid molecules, of known sequence and/or source, by affixing these nucleic acid molecule to particular locations on a support (i.e., features), such as a slide or well bottom, and growing cells that are plated onto the features and maintained under conditions appropriate for entry of the nucleic acid molecules into the cells.

In particular aspects of the invention, we have found that hydrocolloids such as guar gum (and related materials that include pectin, xanthan gum, carrageenan, locust bean gum, tragacanth, gum ghatti, alginates, karaya gum, opopanax, lacquer gutta-percha, mucilage, mesquite gum, kino gum, butea gum, chicle gum, cherry-tree gum, agar-agar, gum albanum, gum olibanum, gum eurphorbium, dragon's blood, conima, gum ammoniac, gutta balata, eucalyptus gum, red gum, and sweet gum) can be used in the production of transfected cell arrays to improve the quality and performance of the arrays. Gums can be added to solutions of transfection complex to significantly improve: a) the stability of the solutions prior to spotting the arrays, b) the process of spotting the solutions on a support, c) the drying of the spots on the support, d) the stability of the dried spots, e) the disposition of the transfection complex during cell culture of the array, and e) the exposure of cells to one or more transfection reagents. More broadly the use of gums may prove advantageous in any transfection/delivery application where materials such as a transfection reagent, nucleic acid, protein, virus, singly or in combination, are mixed with the gums and laid down as a substrate prior to seeding of cells. Gums may also be of value as semi-solid agents for delivery in various applications such as in vivo gene therapy.

Compounds in addition to natural gums which may be included in deposition agents, include synthetic compounds which have properties similar or identical to gums (e.g., are equivalent to natural gums but are produced synthetically), carbohydrates such as agar.

As noted above, hydrocolloids suitable for use in the invention need not be derived from natural sources. Synthetic compounds may also be used. Examples of such synthetic compounds include polybrene, vinyl polymers, polyvinylpyrrolidone, polyvinylalcohol, carboxyvinyl polymer, acrylic polymers, polyacrylic acid, polyacrylamide, ethylene oxide polymers, polyethylenimine (PEI), intact and fractured polyamidoamine dendrimer, asialo-orosomucoid(AsOR)/polylysine, polyamidoamine dendrimer, poly(L-ornithine), DEAE-dextran, gramicidin S, and glycerophosphoethanolamine, as well as derivatives thereof (e.g., fluorinated glycerophosphoethanolamine).

The addition of proteins to the transfection complex has previously been shown to improve the performance of transfected cell arrays (see, e.g., Sabatini et al., U.S. Pat. No. 6,544,790 and Ghadiri et al., U.S. Patent Publication No. 2004/0121333). In particular, gelatin and fibronectin have been successfully used and are thought to aid in the adhesion of cells to the array. We have found that common protein reagents such as albumin can be equally effective.

Our improved methods for preparing arrays from transfection complex mixtures were also tested with lentivirus. Thus, we have successfully prepared viral transfected cell arrays.

III. Overview

The growing collection of genes, cloned cDNAs and functional sub-fragments of genes (e.g., RNAi molecules) has given rise to the development of systematic and high-throughput approaches to characterize function. The uses of DNA microarrays for transcriptional profiling and of two-hybrid assays for determining protein-protein interactions are recent examples of genomic approaches to the characterization of gene products. Reverse transfection arrays have also been developed which allow for the introduction of, for example, nucleic acid molecules into cells and the identification of functional characteristics conferred upon cells by those nucleic acid molecules.

The present invention relates, in part, to improvements in reverse transfection arrays. Some of these improvements relate to the variations in the target bipolymers compositions which are located on supports. For example, nucleic acid molecules which are located on a support may be present in a viral particle or a virus. Additionally, target biopolymers may be co-localized with one or more particular protein, one or more carbohydrate (e.g., one or more gum), or a combination of one or more particular protein (e.g., an albumin such as bovine serum albumin) and one or more carbohydrate. Additionally, one or more transfection reagent may be present in the features.

Nucleic acid molecules which are located on a support may also be localized within a viral particle (e.g., a lentiviral particle, and adenoviral particle, etc.) or a virus. When nucleic acid molecules are localized on a support within a viral particle or a virus, one or more of the following compounds may be co-localized on the support: one or more protein (e.g., a non-viral protein), one or more gum or other deposition agent, one or more transfection reagent, one or more cationic lipid, or one or more anionic lipid.

Features of an array of the invention may also contain non-polymeric molecules, such as small molecules. Small molecule arrays are described in U.S. Patent Publication No. 2003/0032203, the entire disclosure of which is incorporated herein by reference. Small molecules which may be used to prepare arrays of the invention include drug candidates. Often these small molecules will have a molecular weight of less than 1,000.

The invention thus provides, in part, improved transfection arrays. Some improvements provided by the invention include decreased production costs, increased transfection array stability, and increased transfection efficiency.

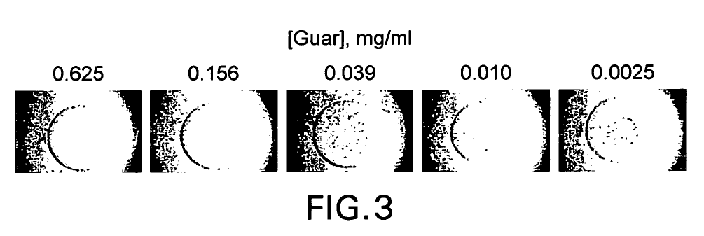

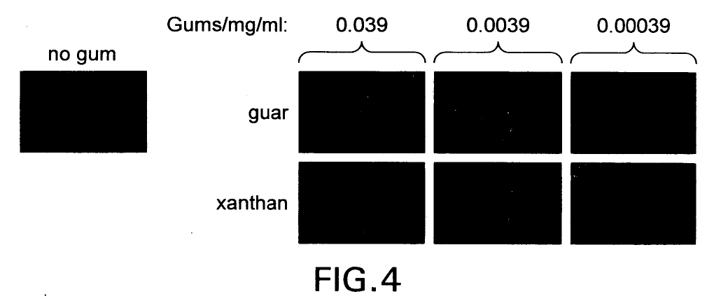

In one aspect, the invention represents a novel application of hydrocolloid gums to transfected cell arrays. One item that we have found is that the addition of gums such as guar to a transfection complex made up of nucleic acid and lipid transfection reagent in buffer offers several distinct improvements to previous reverse transfection array methods. FIG. 3 demonstrates that the addition of guar to the transfection complex solution prior to spotting results in more uniform spots. The presence of gums in the spots also enhanced transfection of cells (FIG. 4).

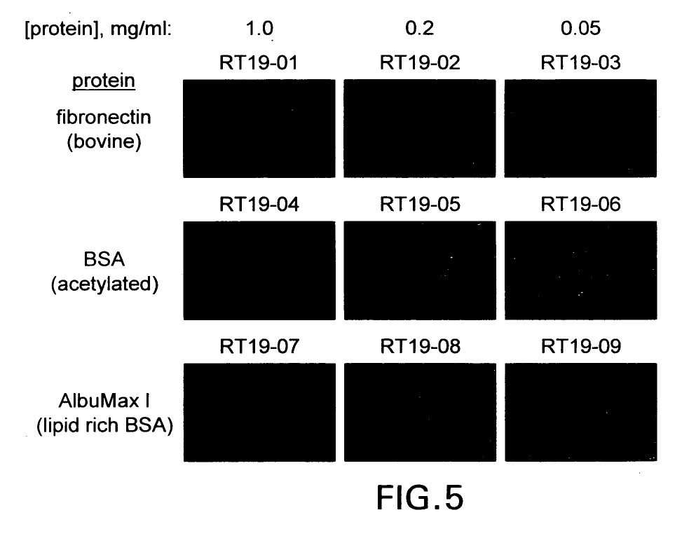

We have also demonstrated that addition of proteins whose physiological functions are unrelated to cell adhesion can also benefit transfected cell arrays. FIG. 5 compares transfection on spots prepared with fibronectin and albumin. The effect of adding protein to the transfection complex may simply be due to the general properties of the protein. Thus, in particular aspect, the invention includes the use of features which contain proteins such as albumin and fibronectin. Typically, the protein(s) present in features will not have a deleterious effect of the target biopolymer(s). For example, if the target biopolymer is a protein, typically, the protein will not have no or little protease activity towards the target biopolymer. Along these lines, in particular embodiments, features of the invention will often contain a protein which with no or little of one or more of the following activities: protease activity (e.g., serine protease activity, etc.), nuclease activity (e.g., DNAase activity and/or RNAse activity, etc.), kinase activity, methylase activity, glycosylation activity, etc.

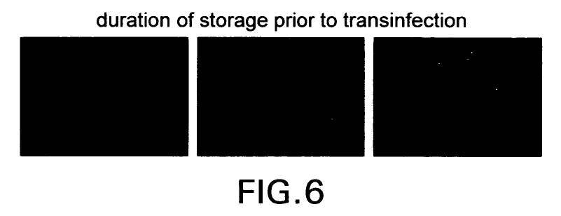

The use of gums can also be extended to viral transfected cell arrays. A mix of lentivirus encoding green fluorescent protein was prepared with sucrose and guar gum, arrayed and seeded with cells. The expression of GFP was observed in the cells, and the viral arrays exhibited a shelf life of over three weeks (FIG. 6.). Infection of cells by lentivirus occurred in the absence of transfection reagent and added protein.

A key advantage of the reverse transfection procedure is the ability to simultaneously screen a cell line with multiple transfection (or infection) vectors. The number of vectors that can be tested on a single array depends on the spot (i.e., feature) density of the array, which is determined by the spot size, the spacing between spots, the array area, and the number of replicates per vector. For example, on Corning GAPS II slide (Corning Inc, Acton, Mass., cat. no. 40003, 40004, 40005, and 40006) the usable surface area is generally around 20 mm×60 mm (12 cm2); with a spot diameter of 0.5 mm (this would accommodate roughly 1,000 cells) and an edge-to-edge spacing of 0.5 mm a 1,200 spots (20×60) array could be accommodated inside this area. This corresponds to a maximal array density of 100 spots/cm2 on standard 1 inch×3 inch slides. Control spots (e.g., positive and negative expression controls, fiduciary spots) and replicate vector spots will determine the total number of unique vectors which may be applied to each array.

IV. Arrays and Array Features

The size of and the quantity (density) of the features on the support can be adjusted depending on the conditions used in the methods. For example, features can be from about 0.1 millimeters (mm) to about 5.0 mm in diameter (e.g., from about 0.1 mm to about 4.5 mm, from about 0.1 mm to about 4.0 mm, from about 0.1 mm to about 3.5 mm, from about 0.5 mm to about 4.5 mm, from about 0.5 mm to about 2.0 mm, from about 0.5 mm to about 1.5 mm, from about 1.0 mm to about 4.5 mm, from about 1.0 mm to about 3.5 mm, from about 1.0 mm to about 3.0 mm, from about 2.0 mm to about 4.5 mm, from about 2.0 mm to about 4.0 mm, etc.) and can be affixed from about 0.05 mm to about 10 mm apart (e.g., from about 0.05 mm to about 8.0 mm, from about 0.05 mm to about 6.0 mm, from about 0.05 mm to about 5.0 mm, from about 0.05 mm to about 4.0 mm, from about 0.05 mm to about 3.0 mm, from about 0.05 mm to about 2.0 mm, from about 0.5 mm to about 8.0 mm, from about 0.5 mm to about 6.0 mm, from about 0.5 mm to about 5.0 mm, from about 0.5 mm to about 4.0 mm, from about 0.5 mm to about 3.0 mm, from about 1.0 mm to about 8.0 mm, from about 1.0 mm to about 6.0 mm, from about 1.0 mm to about 5.0 mm, from about 1.0 mm to about 4.0 mm, from about 2.0 mm to about 8.0 mm, from about 2.0 mm to about 5.0 mm, etc.) measured from edge to edge, on the support. The feature densities and spacings referred to immediately above will typically be characteristics of planar support such as glass microscope slides.

In some instances multi-well plates may be used instead of a planar support. When the support is a multi-well plate (e.g., a 6 well or 96 well plate), the spot size and density may be different than indicated above. When a multi-well plate is used in methods of the invention, the distance between features will typically be determined by the specifications of the plate. Often the distance between features will be in the range of from about 2.0 mm to about 35 mm (e.g., from about 3.0 mm to about 25 mm, from about 5.0 mm to about 30 mm, from about 5.0 mm to about 20 mm, etc.).

The present method further includes identification and/or detection of cells into which target biopolymers or other compounds has been introduced, referred to as “reverse transfection”. In one embodiment, a nucleic acid segment introduced into cells is expressed in the cells, either by an expression vector containing the nucleic acid segment or as a result of integration of the reverse transfected nucleic acid segment into host cell nucleic acid, from which it is expressed.

As explained below, in alternative embodiments of methods of the invention, nucleic acid segments introduced into cells are not expressed, but they affect cell components and/or function itself. For example, double-stranded RNA can be introduced into cells and alter one or more cell function. For example, double-stranded RNA which mediates RNA interference driven degradation of a mRNA encoding a receptor for a drug can be introduced into cells via reverse transfection. The double-stranded RNA may knock-down expression of the drug receptor protein, causing a decrease in drug binding to cells containing the double-stranded RNA.

In particular methods of the invention, a mixture comprising a nucleic acid molecule of interest (such as cDNA or genomic DNA incorporated in an expression vector) and a deposition agent is deposited (e.g., spotted or placed in small defined areas) onto a surface of a support (e.g., a slide or other flat surface, such as the bottoms of wells in a multi-welled plate) in defined, discrete (distinct) locations and allowed to dry, with the result that the DNA-containing mixture is affixed to the surface in defined, discrete locations.

Detection of effects (either direct or indirect effects) on recipient cells (cells containing nucleic acid molecules introduced by reverse transfection) can be carried out by a variety of known techniques, such as immunofluorescence, in which a fluorescently labeled antibody that binds a protein of interest (e.g., a protein thought to be encoded by a reverse transfected nucleic acid molecule or a protein whose expression or function is altered through the action of the reverse transfected nucleic acid molecule) is used to determine if the protein is present in cells grown on the features.

Methods of this invention are useful, for example, to identify nucleic acid molecules of interest (e.g., DNAs that are expressed in recipient cells or act upon or interact with recipient cell constituents or function, such as DNAs that encode a protein whose function is desired because of characteristics its expression gives cells in which it is expressed).

Target biopolymers can be used in a variety of formats, including macro-arrays and micro-arrays and permit, for example, a nucleic acid array to be converted into a protein or cell array, such as a protein or cell microarray. In other words, once cells have been transfected with features which contain different biopolymers, the

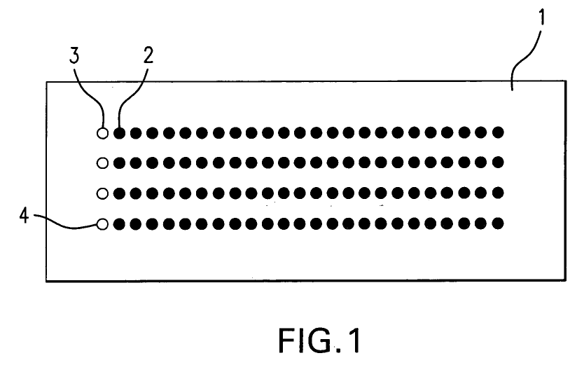

An exemplary format for an array of the invention is shown in FIG. 1. In this instance, the molecular array has been printed on a glass microscope slide. This array (1) contains 96 test features (2), 3 control features (3), and a blank (4). The control features contain a target biopolymer or other molecules which can be either directly or indirectly detected after entry into cells (3). The blank (4) may contain the deposition agent and/or any other compounds in other features but does not contain the target biopolymer or other compound to be transfected into cells.

Each feature of arrays of the invention will typically contain at least one target biomolecule or other compound (e.g., a drug candidate) which one desires to get into cells.

In many instance, features will contain nucleic acid molecules. These nucleic acid molecules (e.g., RNA, DNA, etc.) may vary greatly in their origin and structure. As examples, nucleic acid molecules used in the practice of the invention include expression vectors, homologous recombination cassettes, and double-stranded RNA molecules.

As noted above, nucleic acid molecules contained in features may be designed to exert either a direct or indirect effect upon cells into which they enter. An exemplary nucleic acid molecule which may be present in one or more features of an array of the invention is an expression vector. Expression vectors may be designed to generate mRNAs or functional RNAs (e.g., tRNAs, ribozymes, double-stranded RNAs which mediated RNA interference, etc.).

In certain embodiments, individual features may contain individual members of a library of expression vectors. Libraries can be produced by ligating a polynucleotide coding sequence or other transcribable sequences into a cloning site of an expression vector using standard procedures. Similar procedures, or modifications thereof, can be readily employed to prepare arrays of expression vectors in accord with the subject invention.

In certain embodiments, features may contain members of a library of related, mutated sequences, such as a library of mutants of a particular protein, or libraries of potential promoter sequences, etc. There are a variety of forms of mutagenesis that can be utilized to generate a combinatorial library. For example, homologs of protein of interest (both agonist and antagonist forms) can be generated and isolated from a library by screening using, for example, alanine scanning mutagenesis and the like (Ruf et al. (1994) Biochemistry 33:1565-1572; Wang et al. (1994) J. Biol. Chem. 269:3095-3099).

In other embodiments, features contain members of a library of small gene fragments (e.g., nucleic acid segments which may encode dominant-acting synthetic genetic elements (SGEs) (e.g., molecules that interfere with the function of genes from which they are derived (antagonists) or that are dominant constitutively active fragments (agonists) of such genes)). SGEs that can be identified by methods of the invention include, but are not limited to, inhibitory antisense RNA molecules, ribozymes, nucleic acid decoys, double-stranded RNAs which mediate RNA interference, and nucleic acid segments which encode polypeptides.

SGEs identified by methods of the invention may function to inhibit the function of an endogenous gene at the level of nucleic acids (e.g., by an antisense or decoy mechanism), or by encoding a polypeptide that is inhibitory through a mechanism of interference at the protein level (e.g., a dominant negative fragment of the native protein). On the other hand, certain SGEs may function to potentiate (including mimicking) the function of an endogenous gene by encoding a polypeptide which retains at least a portion of the bioactivity of the corresponding endogenous gene, and may in particular instances be constitutively active.

In one embodiment, an initial SGE library is generated from total cDNA, that may be further fragmented, and provided in the form of an expression library. In many instances, the inserts in the library will range from about 100 bp to about 700 bp or from about 200 bp to about 500 bp in size.

For cDNA-derived libraries, the nucleic acid library may be a normalized library containing roughly equal numbers of clones corresponding to each gene expressed in the cell type from which it was made, without regard for the level of expression of any gene.

U.S. Pat. No. 5,702,898 describes a method to normalize a cDNA library constructed in a vector capable of being converted to single-stranded circles and capable of producing complementary nucleic acid molecules to the single-stranded circles comprising: (a) converting the cDNA library into single-stranded circles; (b) generating complementary nucleic acid molecules to the single-stranded circles; (c) hybridizing the single-stranded circles converted in step (a) with complementary nucleic acid molecules of step (b) to produce partial duplexes to an appropriate Cot; and (d) separating the unhybridized single-stranded circles from the hybridized single-stranded circles, thereby generating a normalized cDNA library.

Libraries used in the practice of the invention may be generated to include both sense and antisense coding (and non-coding sequences) sequences. Further, in certain embodiments, these libraries may be subtractive cDNA libraries. Many strategies have been used to create subtractive libraries, and can be readily adapted for use in the practice of the invention. One approach is based on the use of directionally cloned cDNA libraries as starting material (Palazzolo and Meyerowitz, (1987) Gene 52:197; Palazzolo et al. (1989) Neuron 3:527; Palazzolo et al. (1990) Gene 88:25). In this approach, cDNAs prepared from a first source tissue or cell line are directionally inserted immediately downstream of a bacteriophage T7 promoter in the vector. Total library DNA is prepared and transcribed in vitro with T7 RNA polymerase to produce large amounts of RNA that correspond to the original mRNA from the first source tissue. Sequences present in both the source tissue and another tissue or cells, such as normal tissue, are subtracted as follows. The in vitro transcribed RNA prepared from the first source is allowed to hybridize with cDNA prepared from either native mRNA or library RNA from the second source tissue. The complementarity of the cDNA to the RNA makes it possible to remove common sequences as they anneal to each other, allowing the subsequent isolation of unhybridized, presumably tissue-specific, cDNA.

In other embodiments, features contain members of a library encoding a variegated population of small peptides (e.g., 4-25 amino acid residues in length). This library may be generated from coding sequences of total cDNA, or single genes, or can be random or semi-random in sequence. Small peptide fragments, corresponding to only a minute portion of a protein, can inhibit the function of that protein in vivo.

In additional aspects of the invention, one or more feature of an array of the invention may contain two or more target biopolymers. The target biopolymers of these features may be characterized, partially characterized, or uncharacterized (e.g., the nucleotide sequence of the nucleic acid intended for expression is not known). By partially characterized what is meant is that after one target biopolymer in a feature has been characterized, characteristics of this one target biopolymers are then known. Thus, as work with an array in this aspect of the invention is performed, target biopolymers of features become characterized. Further, when known target biopolymers are mixed in a feature, then the target biopolymers may be said to be characterized.

As an example of this aspect of the invention, nucleic acid molecules of a cDNA library (e.g., a normalized library, a full-length library, etc.) may be amplified in bacterial cells, the amplified nucleic acid molecules from a number of clonal isolates of the bacterial cells may then be placed in the same feature of the array. This process may then be repeated for other features.

Cells (e.g., eukaryotic cells) may be contacted with an array described above under conditions which allow for uptake of the nucleic acid molecules by the contacted cells. Cells which express one or more specific phenotype may then be identified. The location(s) of nucleic acid molecules on the array which confer a particular phenotype upon cells in which it they are expressed may be identified by transfecting a population of cells and identifying cells which have the particular phenotype.

In one specific example, a microarray prepared on a glass slide is prepared with a format as essentially shown in FIG. 1. The non-control features (2) each contain 20 different vectors which are designed to express different members of a human cDNA library. In particular, the nucleic acid of the cDNA library members are operably linked to a CMV promoter. Further, the features contain, in addition to the cDNA library members a transfection reagent (e.g., Lipofectamine™ 2000) and gelatin. This aspect of the invention, as well as other aspects of the invention, is not limited to the use of particular deposition agents.

After the above microarray has been produced, human cells which do not normally express the protein PD-L2 are then contacted with the features and cells which exhibit the presence of PD-L2 on the cells surface are identified using fluorescently labeled anti-PD-L2 antibodies (see He et al., Acta. Biochim. et. Biophysica Sinica, 36(4):284-289 (2004)). Once cells which express PD-L2 are identified, the location of the feature(s) which conferred the PD-L2 plus phenotype may then be identified. These features will typically contain either the PD-L2 gene itself or will encode a molecules which either directly or indirectly leads to the PD-L2 production. In instances, where the original nucleic acid molecules of the features (1) have been stored or (2) can be retrieved from either the cells or the features themselves, it will be possible to obtain the twenty nucleic acid molecules of the feature. Typically, these methods of the invention will be designed to narrow the number of candidate nucleic acid molecules which confer a particular phenotype, with the ultimate goal of identifying a single nucleic acid molecule which actually confers the particular phenotype.

Using methods described above, it is possible to place a large number of target biopolymers on a small number of arrays (e.g., one array). This allows for convenient screening of a large number of target biopolymers to reduce the number of molecules which confer particular characteristics upon cells. Typically, further screening will be done to identify a specific target biopolymer which confers one or more particular characteristic upon the cells.

As one skilled in the art would understand, the features of arrays described above may contain any number of different target biopolymers (e.g., two, three, four, five, six, seven, eight, nine, ten, twelve, fifteen, eighteen, twenty, thirty, fifty, one hundred, two hundred, etc.). One or more feature of an array of the invention may contain different target biopolymers in the following ranges: from about 2 to about 200, from about 2 to about 100, from about 2 to about 50, from about 2 to about 25, from about 10 to about 200, from about 10 to about 100, from about 10 to about 50, from about 10 to about 25, from about 20 to about 200, from about 20 to about 100, from about 20 to about 75, from about 20 to about 50, from about 40 to about 200, from about 40 to about 150, from about 40 to about 100, from about 40 to about 75, from about 50 to about 200, from about 50 to about 150, from about 50 to about 100, etc.

Additionally, the number of different target biopolymers in each non-control feature may be the same or different. Thus, when 50 non-control features are present, 10 may contain 32 different target biopolymers, 12 may contain 30 different target biopolymers, 9 may contain 25 different target biopolymers, 10 may contain 22 different target biopolymers, and 9 may contain 18 different target biopolymers.

Target biopolymers may also be clustered in features by one or more related functions. For example, again using as an example an array where 50 non-control features are present, four features may respectively contain nucleic acid molecules which encode 8, 12, 13, and 15 different kinases; three features may respectively contain nucleic acid molecules which encode 4, 6, and 12 different proteinases; three features may respectively contain nucleic acid molecules which encode 8, 8, and 8 different peptidases; three features may respectively contain nucleic acid molecules which encode 3, 5, and 7 different nucleases, etc.

Similar to the above, features may contain different target biopolymers which have relatively unrelated functions. For example, a feature may contain 6 different nucleic acid molecules which encode kinases and 5 different nucleic acid molecules which encode phosphates. When a particular cellular effect is conferred by such a feature, 5 new features may be generated and screened which contain the 6 different nucleic acid molecules which encode kinases, each feature containing one of the different nucleic acid molecules which encode phosphatase. If one of the features which contains a nucleic acid molecule which encode phosphatase is shown to confer the particular cellular effect, then 6 additional features may be generated which contain nucleic acid molecules which encode phosphatase of interest and each of the individual nucleic acid molecules which encode kinases.

The approach of the invention set out above allows for combinatorial scale screening of target biopolymers to identify target biopolymers which interact with each other to confer cellular effects or phenotypes. The invention thus includes iterative screening processes for identifying interactive cellular effects of target biopolymers.

Aspects of the invention referred to above are particular suited to business methods of the invention. In particular, the product supplied to a customer may be an array. The customer then uses the array to identify features which confer one or more particular phenotypes upon cells of interest. After identification of these features, the customer may then order either the individual nucleic acid molecules present in the features or mixtures of nucleic acid molecules which are present in the features. The customer may then introduce the nucleic acid molecules into cells and specifically identify nucleic acid molecules which confer the particular phenotypes.

As noted elsewhere herein, in many instances, it will be desirable that the vector be capable of replication in the host cell into which it is introduced. The vector may be DNA which is integrated into the host genome, and thereafter is replicated as a part of the chromosomal DNA, or it may be DNA which replicates autonomously, as in the case of a episomal plasmid. In the latter case, the vector may include an origin of replication which is functional in the host. In the case of an integrating vector, the vector may include sequences which facilitate integration (e.g., sequences homologous to host sequences, or encoding integrases). The use of retroviral long terminal repeats (LTR) or adenoviral inverted terminal repeats (ITR) in the construct of the transfection array can, for example, facilitate the chromosomal integration of the construct.

A considerable number of expression vectors are known in the art and numerous additional expression vectors can be constructed. As noted above, typically, expression vectors will contain an insertion site into which nucleic acid intended for expression can be introduced and a transcription regulatory sequence operably connected to the insertion site. If the expression is intended to be stably maintained in cells, then it will often additionally contain a selection marker and an origin of replication which functions in the cell into which it is introduced (e.g., a cell which is contacted with a feature which contains the expression vector). In many instance, for transient transfection, a selection marker and functional origin of replication will not be required.

As also noted above, expression vectors useful in the present invention include chromosomal-, episomal-vectors. After insertion into such an expression vector, the nucleic acid insert will often be operatively linked to an appropriate promoter, such as the MMTV promoter, metalothionine promoter, RSV promoter, SV40 promoter, hGH promoter, CMV promoter, U6 promoter, H1 promoter, and ubiquitin promoter. Other suitable promoters will be known to the skilled artisan. Thus, promoters used in the practice of the invention include RNA polymerase I, II, or III promoters.

Vectors which may be used in the invention include pQE70, pQE60 and pQE-9, available from Qiagen; pBS vectors, Phagescript vectors, Bluescript vectors, pNH8A, pNH16a, pNH18A, pNH46A, available from Stratagene; pcDNA3 available from Invitrogen Corporation (Carlsbad, Calif.); pGEX, pTrxfus, pTrc99a, pET-5, pET-9, pKK223-3, pKK233-3, pDR540, pRIT5 available from Pharmacia; and Gateway™ vectors such as pET-DEST42, pDESTTM™14, pBAD-DEST49, pYES-DEST52, pDEST™ 8, pcDNA-DEST40, pT-REx-DEST30, pENTR 1A, pENTR 2B, pENTR3C, pDONR™ 201, pDONR™ 207, and other vectors such as pSPORT1, pSPORT2 and pSV.SPORT1, pSPORT6, available from Invitrogen Corporation (Carlsbad, Calif.). Other suitable vectors will be readily apparent to the skilled artisan and include the mammalian expression vectors pcDNAI/amp, pcDNAI/neo, pRc/CMV, pSV2gpt, pSV2neo, pSV2-dhfr, pTk2, pRSVneo, pMSG, pSVT7, pko-neo and pHyg, as well as additional vectors derived from these vectors.

Additional, nucleic acid molecules used in the practice of the invention may contain one or more (e.g., one, two, three, four, etc.) recombination site or one or more topoisomerase recognition site. Typically, these recombination sites and/or topoisomerase recognition sites will be employed to construct nucleic acid molecules which reside in features.

Cloning systems employing recombination sites and topoisomerases are well know in the art and include Invitrogen Corporation's Gateway™ and topoisomerase cloning systems (see, e.g., Invitrogen Corp., Carlsbad, Calif. cat. nos. 12535-019, 12535-027, 12535-035, 11798-014, K4530-20, and KNM4600-01).

Recombination sites which may be present in nucleic acid molecules used in various aspects of the invention include att sites (e.g., attB sites, attL sites, attR sites, attP sites, etc.), lox sites (e.g., loxP sites), psi sites, dif sites, cer sites, and frt sites.

Nucleic acid molecules present in features may vary in size from 20 nucleotides or base pairs 2,000,000 nucleotides or base pairs. The sizes of nucleic acid molecules used in the practice of the invention will often depend upon the particular application. As an example, when the nucleic acid molecules are intended to knock-down gene expression by RNA interference in higher eukaryotic cells, the molecules will typically be double-stranded molecules which are less than 30 base pairs in length. As evidenced by their size, such nucleic acid molecules will typically not be capable of replicating once in cells.

Nucleic acid molecules used in the practice of the invention may be either incapable or capable of self replicating. Examples of nucleic acid molecules which are capable of self replication include plasmids. Nucleic acid molecules which are not capable of self replicating may still be replicated once introduced into cells. For example, such molecules may integrate into host cell nucleic acids and be replicated along with these host cell nucleic acids.

Other nucleic acid molecules which are not capable of self replicating will typically not be replicated when introduced into cells. Examples of such nucleic acid molecules include double-stranded RNA or chemically modified nucleic acid molecules, which may be single-stranded or double-stranded. Further, double-stranded nucleic acid molecules may be designed to mediate RNA inference in cells. Typically such molecules do not contain origins of replication and do not integrate into nucleic acids present in host cells.

One example of a chemically modified nucleic acid molecule which may be used in the practice of the invention is Stealth™ (Invitrogen Corp., Carlsbad, Calif.).

Any suitable promoter may be used to control the production of RNA from the nucleic acid molecules of the invention. Promoters may be those recognized by any polymerase enzyme. For example, promoters may be promoters for RNA polymerase II or RNA polymerase III (e.g., a U6 promoter, an H1 promoter, etc.). Other suitable promoters include, but are not limited to, T7 promoter, cytomegalovirus (CMV) promoter, mouse mammary tumor virus (MMTV) promoter, metalothionine, RSV (Rous sarcoma virus) long terminal repeat, SV40 promoter, human growth hormone (hGH) promoter. Other suitable promoters are known to those skilled in the art and are within the scope of the present invention.

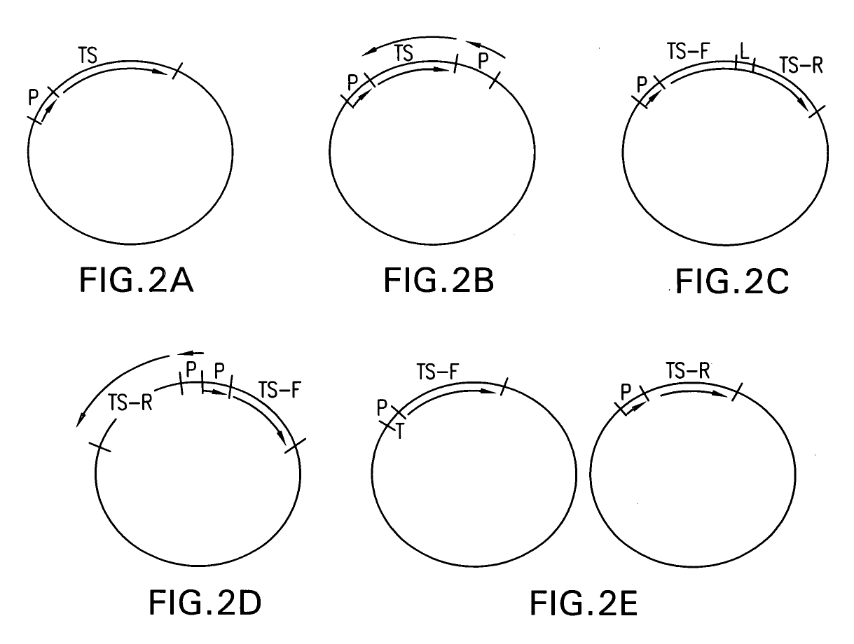

One example of a construct designed to produce RNAi is shown in FIG. 2B. In this construct, a DNA segment is inserted into a vector such that RNA corresponding to both strands are produced as two separate transcripts. Another example of a construct designed to produce RNAi is shown in FIG. 2C. In this construct, two copies of a DNA segment are inserted into a vector such that RNA corresponding to both strands are produced as a single transcript which will hybridize intramolecularly to produce a “hairpin” RNA molecule. Yet another example of a construct designed to produce RNAi is shown in FIG. 2D. In this construct, two copies of a DNA segment are inserted into a vector such that RNA corresponding to both strands are again produced. The exemplary vector system shown in shown in FIG. 2E comprises two vectors, each of which contain copies of the same DNA segment. Expression of one of these DNA segments results in the production of sense RNA while expression of the other results in the production of an anti-sense RNA. RNA strands produced from vectors represented in FIGS. 2B-2E will thus have complementary nucleotide sequences and will generally hybridize either to each or intramolecularly under physiological conditions.

Nucleic acid segments designed to produce RNAi, such as the vectors represented in FIGS. 2B-2E, need not correspond to the full-length gene or open reading frame. For example, when the nucleic acid segment corresponds to an ORF, the segment may only correspond to part of the ORF (e.g., 25 nucleotides at the 5′ or 3′ end of the ORF). Further, while FIGS. 2B-2E show vectors designed to produce RNAi, nucleic acid segments may also perform the same function in other forms (e.g., when inserted into the chromosome of a host cell).

Gene silencing methods involving the use of compounds such as RNAi and antisense RNA, for examples, are particularly useful for identifying gene functions. More specifically, gene silencing methods can be used to reduce or prevent the expression of one or more genes in a cell or organism. Phenotypic manifestations associated with the selective inhibition of gene functions can then be used to assign role to the “silenced” gene or genes. As an example, Chuang, et al., Proc. Natl. Acad. Sci. (USA) 97:4985-4990 (2000), have demonstrated that in vivo production of RNAi can alter gene activity in Arabidopsis thaliana. Thus, the invention provides methods for regulating expression of nucleic acid molecules in cells and tissues comprising the expression of RNAi and antisense RNA. The invention further provides methods for preparing nucleic acid molecules which can be used to produce RNA corresponding to one or both strands of a DNA molecule.

Any number of different viral vectors, viral particles and/or viruses may be used in the practice of the invention. For example, nucleic acid molecules contained within lentiviral particles and/or viruses may be placed on supports. These supports may then be contacted with cells. One advantage to using lentiviral vectors is that they can transfect non-dividing cells.

Recombinant viruses are currently used in wide variety of applications. Examples of recombinant viruses that have been used include, but are not limited to, herpes viruses (see, for example, U.S. Pat. No. 5,672,344), pox viruses such as vaccinia virus (see, for example, Moss, et al., 1997, in Current Protocols in Molecular Biology, Chapters 16.15-16.18, John Wiley & Sons), papilloma viruses (see, for example, U.S. Pat. No. 6,342,224), retroviruses (see, for example U.S. Pat. No. 6,300,118), adenoviruses (see, for example, U.S. Pat. No. 6,261,807), adeno-associated viruses (AAV, see for example, U.S. Pat. No. 5,252,479), and coxsackie viruses (see, for example, U.S. Pat. No. 6,323,024).

When the viral vector nucleic acid is not infectious, construction of recombinant viruses may involve in vivo homologous recombination in a virus-infected cell between the viral vector and concomitantly transfected plasmid bearing a sequence of interest flanked by viral sequences. When the viral nucleic acid is infectious, a modified viral nucleic acid may be prepared and transfected into a host cell. Either non-infectious or infectious viral nucleic acid may be used in the practice of the invention. For example, it may be advantageous to use non-infectious viral nucleic acid when one seeks to obtain cells which have undergone homologous recombination with the viral vector.

Adenoviruses are non-enveloped viruses with a 36 kb DNA genome which encodes more than 30 proteins. At the ends of the genome are inverted terminal repeats (ITRs) of approximately 100-150 base pairs. A sequence of approximately 300 base pairs located next to the 5′-ITR is required for packaging of the genome into the viral capsid. The genome as packaged in the virion has terminal proteins covalently attached to the ends of the linear genome.

The genes encoded by the adenoviral genome are divided into early and late genes depending upon the timing of their expression relative to the replication of the viral DNA. The early genes are expressed from four regions of the adenoviral genome termed E1-E4 and are transcribed prior to onset of DNA replication. Multiple genes are transcribed from each region. Portions of the adenoviral genome may be deleted without affecting the infectivity of the deleted virus. The genes transcribed from regions E1, E2, and E4 are essential for viral replication while those from the E3 region may be deleted without affecting replication. The genes from the essential regions can be supplied in trans to allow the propagation of a defective virus. For example, deletion of the E1 region of the adenoviral genome results in a virus that is replication defective. Viruses deleted in this region are grown on 293 cells that express the viral E1 genes from the genome of the cell. Nucleic acid molecules used in the practice of the invention may contain one or more adenoviral genetic element referred to above (e.g., one or more adenoviral early gene, one or more adenoviral packaging signal, one or more ITR, etc.).

In addition to permitting the construction of a safer, replication-defective viruses, deletion and complementation in trans of portions of the adenoviral genome and/or deletion of non-essential regions make space in the adenoviral genome for the insertion of heterologous DNA sequences. The packaging of viral DNA into a viral particle or virus is often size restricted with, in many instances, an upper limit of approximately 38 kb of DNA. In order to maximize the amount of heterologous DNA that may be inserted and packaged, viruses have been constructed that lack all of the viral genome except the ITRs and packaging sequence (see U.S. Pat. No. 6,228,646). All of the viral functions necessary for replication and packaging are provided in trans from a defective helper virus that is deleted in the packaging signal.

While any number of adenoviral vectors may be used in the practice of the invention, one specific adenoviral system is the ViraPower™ Adenoviral Expression System available from Invitrogen Corporation, Carlsbad, Calif. (see, e.g., cat. nos. K4930-00 and K4940-00). In particular embodiments of the invention, adenoviral particles are deposited on supports (e.g., solid supports) and then contacted with cells.

Baculoviral vectors may also be used in the practice of the present invention. Baculoviruses are large, enveloped viruses which typically infect arthropods. Baculoviral genomes are double-stranded DNA molecules of approximately 130 kbp in length. Baculoviruses have gained widespread use as systems in which to express proteins, particularly proteins from eukaryotic organisms (e.g., mammals), as the insect cells used to culture the virus may more closely mimic the post-translational modifications (e.g., glycosylation, acylation, etc.) of the native organism.

Numerous expression systems utilizing recombinant baculoviruses have been developed. General methods for constructing recombinant baculoviruses for expression of heterologous proteins may be found in Piwnica-Worms, et al., (1997) Expression of Proteins in Insect Cells Using Baculovirus Vectors, in Current Protocols in Molecular Biology, Chapter 16, pp. 16.9.1 to 16.11.12, Ausubel, et al. Eds., John Wiley & Sons, Inc. Other expression systems are known, for example, U.S. Pat. No. 6,255,060 discloses a baculoviral expression system for expressing nucleotide sequences that include a tag. U.S. Pat. No. 5,244,805 discloses a baculoviral expression system that utilizes a modified promoter not naturally found in baculoviruses. U.S. Pat. No. 5,169,784 discloses a baculoviral expression system that utilizes dual promoters (e.g., a baculoviral early promoter and a baculoviral late promoter). U.S. Pat. No. 5,162,222 discloses a baculoviral expression system that can be used to create stable cells lines or infectious viruses expressing heterologous proteins from a baculoviral immediate-early promoter (i.e., IEN). U.S. Pat. No. 5,155,037 discloses a baculoviral expression system that utilizes insect cell secretion signal to improve efficiency of processing and secretion of heterologous genes. U.S. Pat. No. 5,077,214 discloses the use of baculoviral early gene promoters to construct stable cell lines expression heterologous genes. U.S. Pat. No. 4,879,239 discloses a baculoviral expression system that utilizes the baculoviral polyhedrin promoter to control the expression of heterologous genes.

Various methods of constructing recombinant baculoviruses have been used. A frequently used method involves transfecting baculoviral DNA and a plasmid containing baculoviral sequences flanking a heterologous sequence. Homologous recombination between the plasmid and the baculoviral genome results in a recombinant baculovirus containing the heterologous sequences. This results in a mixed population of recombinant and non-recombinant viruses. Recombinant baculoviruses may be isolated from non-recombinant by plaque purification. Viruses produced in this fashion may require several rounds of plaque purification to obtain a pure strain. Methods to reduce the background of non-recombinant viruses produced by homologous recombination methods have been developed. For example, a linearized baculoviral genome containing a lethal deletion, BaculoGold™, is commercially available from BD Biosciences, San Jose, Calif. The lethal deletion is rescued by homologous recombination with plasmids containing baculoviral sequences from the polyhedrin locus.

Methods utilizing direct insertion of foreign sequences into a baculoviral genome are also known. For example, Peakman, et al. (Nucleic Acids Res 20(3):495-500, 1992) disclose the construction of baculoviruses having a lox site in the genome. Heterologous sequences may be moved into the genome by in vitro site-specific recombination between a plasmid having a lox site and the baculoviral genome in the presence of Cre recombinase. U.S. Pat. No. 5,348,886 discloses a baculoviral expression system that utilizes a bacmid (a hybrid molecule comprising a baculoviral genome and a prokaryotic origin of replication and selectable marker) containing a recombination site for Tn7 transposon. Prokaryotic cells carrying the bacmid are transformed with a plasmid having a Tn7 recombination site and with a plasmid expressing the activities necessary to catalyze recombination between the Tn7 sites. Heterologous sequences present on the plasmid are introduced into the bacmid by site-specific recombination between the Tn7 sites. The recombinant bacmid may be purified from the prokaryotic host and introduced into insect cells to initiate an infection. Recombinant viruses carrying the heterologous sequence are produced by the cells transfected with the bacmid.

While any number of baculoviral vectors may be used in the practice of the invention, one specific adenoviral system is the Baculovirus Expression System available from Invitrogen Corporation, Carlsbad, Calif. (see, e.g., cat. no. 11827-011). In particular embodiments of the invention, baculoviral vectors are deposited on supports (e.g., solid supports) and then contacted with cells.

Additional examples of baculovirus expression systems which may be used in the practice of the invention include pVL-derived vectors (such as pVL1392, pVL1393 and pVL941), pAcUW-derived vectors (such as pAcUW 1), and pBlueBac-derived vectors (such as the beta-gal containing pBlueBac III). Such systems may be used when it is desirable to introduce target biopolymers into insect cells.

The family Retroviridae contains three subfamilies: 1) oncovirinae; 2) spumavirinae; and 3) lentivirinae. Retroviruses (e.g., lentiviruses) are viruses having an RNA genome that replicate through a DNA intermediate. A retroviral particle typically contains two copies of the RNA genome and viral replication enzymes in a RNA-protein viral core. The core is surrounded by a viral envelop made up of virally encoded glycoproteins and host cell membrane. In the early steps of infection, retroviruses deliver the RNA-protein complex into the cytoplasm of the target cell. The RNA is reverse transcribed into double-stranded cDNA and a pre-integration complex containing the cDNA and the viral factors necessary to integrate the cDNA into the target cell genome is formed. The complex migrates to the nucleus of the target cell and the cDNA is integrated into the genome of the target cell. As a consequence of this integration, the DNA corresponding to the viral genome (and any heterologous sequences contained in the viral genome) is replicated and passed on to daughter cells. This makes it possible to permanently introduce heterologous sequences into cells.

A wide variety of retroviruses are known, for example, leukemia viruses such as a Moloney Murine Leukemia Virus (MMLV) and immunodeficiency viruses such as the Human Immunodeficiency Virus (HIV). Representative examples of retroviruses include, but are not limited to, the Gibbon Ape Leukemia virus (GALV), Avian Sarcoma-Leukosis Virus (ASLV), which includes but is not limited to Rous Sarcoma Virus (RSV), Avian Myeloblastosis Virus (AMV), Avian Erythroblastosis Virus (AEV) Helper Virus, Avian Myelocytomatosis Virus, Avian Reticuloendotheliosis Virus, Avian Sarcoma Virus, Rous Associated Virus (RAV), and Myeloblastosis Associated Virus (MAV).

Retroviruses have found widespread use as gene therapy vectors. To reduce the risk of transmission of the gene therapy vector, gene therapy vectors have been developed that have modifications that prevent the production of replication competent viruses once introduced into a target cell. For example, U.S. Pat. No. 5,741,486 describes retroviral vectors comprising direct repeats flanking a sequence that is desired to be deleted (e.g., a cis-acting packing signal) upon reverse transcription in a host cell. Deletion of the packing signal prevents packaging of the recombinant viral genome into retroviral particles, thus preventing spread of retroviral vectors to non-target cells in the event of infection with replication competent viruses. U.S. Pat. Nos. 5,686,279, 5,834,256, 5,858,740, 5,994,136, 6,013,516, 6,051,427, 6,165,782, and 6,218,187 describe a retroviral packaging system for preparing high titer stocks of recombinant retroviruses. Plasmids encoding the retroviral functions required to package a recombinant retroviral genome are provided in trans. The packaged recombinant retroviral genomes may be harvested and used to infect a desired target cell.

While any number of retroviral vectors may be used in the practice of the invention, one specific adenoviral system is the ViraPower™ Lentiviral Expression System available from Invitrogen Corporation, Carlsbad, Calif. (see, e.g., cat. nos. K4950-00 and K4960-00). In particular embodiments of the invention, lentiviral vectors are deposited on supports (e.g., solid supports) and then contacted with cells.

The family Herpesviridae contains three subfamilies 1) alphaherpesvirinae, containing among others human herpesvirus 1; 2) betaherpesvirinae, containing the cytomegaloviruses; and 3) gammaherpesvirinae. Herpesviruses are enveloped DNA viruses. Herpesviruses form particles that are approximately spherical in shape and that contain one molecule of linear dsDNA and approximately 20 structural proteins. Numerous herpesviruses have been isolated from a wide variety of hosts. For example, U.S. Pat. No. 6,121,043 describes recombinant herpesvirus of turkeys comprising a foreign DNA inserted into a non-essential region of the herpesvirus of turkeys genome; U.S. Pat. No. 6,410,311 describes recombinant feline herpesvirus comprising a foreign DNA inserted into a region corresponding to a 3.0 kb EcoRI-SalI fragment of a feline herpesvirus genome, U.S. Pat. No. 6,379,967 describes herpesvirus saimiri, (HVS; a lymphotropic virus of squirrel monkeys) as a viral vector; and U.S. Pat. No. 6,086,902 describes recombinant bovine herpesvirus type 1 vaccines.

Herpesviruses have been used as vectors to deliver exogenous nucleic acid material to a host cell. In addition to the examples above, U.S. Pat. No. 4,859,587 describes recombinant herpes simplex viruses, vaccines and methods, U.S. Pat. No. 5,998,208 describes a helper virus-free herpesvirus vector packaging system, U.S. Pat. No. 6,342,229 describes herpesvirus particles comprising fusion protein and their preparation and use and U.S. Pat. No. 6,319,703 describes recombinant virus vectors that include a double mutant herpesvirus such as an herpes simplex virus-1 (HSV-1) mutant lacking the essential glycoprotein gH gene and having a mutation impairing the function of the gene product VP 16.

RNA viruses, such as those of the families Flaviviridae and Togaviridae have also been used to deliver exogenous nucleic acids to target cells. For example, members of the genus alphavirus in the family Togaviridae have been engineered for the high level expression of heterologous RNAs and polypeptides (Frolov et al., Proc. Natl. Acad. Sci. U.S.A. 93:11371-11377 (1996)). Alphaviruses are positive stranded RNA viruses. A single genomic RNA molecule is packaged in the virion. RNA replication occurs by synthesis of a full-length minus strand RNA intermediate that is used as a template for synthesis of positive strand genomic RNA as well for synthesis of a positive strand sub-genomic RNA initiated from an internal promoter. The sub-genomic RNA can accumulate to very high levels in infected cells making alphaviruses attractive as transient expression systems. Examples of alphaviruses are Sindbis virus and Semliki Forest Virus. Kunjin virus is an example of a flavivirus. Sub-genomic replicons of Kunjin virus have been engineered to express heterologous polypeptides (Khromykh and Westaway, J. Virol. 71:1497-1505 (1997)). The genomic RNA of both flaviviruses and togaviruses are infectious; transfection of the naked genomic RNA results in production of infective virus.

The invention thus includes arrays with features which include viral particles and/or viruses, as well as uses of these arrays in methods for introducing the viral particles and/or viruses into cells.

Target biopolymers may be arrayed in an addressable fashion, such as rows and columns where the substrate is a planar surface. As noted above, one exemplary format is shown in FIG. 1.

As noted above, in certain embodiments, features may contain multiple (e.g., two, three, four, five, six, seven, eight, nine, ten, etc.) different target biopolymers in each feature (e.g., in order to promote co-transfection of the host cells with at least two different target sequences). Co-transfection refers to the simultaneous introduction of two or more target biopolymers into the same cell. For example, if the target biopolymers are plasmids or other nucleic acid constructs direct the expression of different gene product (e.g., different proteins, RNAs or other gene products), then the cell may express both gene products at the same time.

Co-transfection of cells has several uses. These include, but are not limited to, the ability to: (1) infer the expression of a gene product by detecting the expression of a co-transfected plasmid encoding a marker protein (e.g., GFP, luciferase, beta-galactosidase, or any protein to which a specific antibody is available), (2) express all the components of a multi-subunit complex (e.g., a T-cell receptor) in the same cells, (3) express all the components of a pathway (e.g., a signal transduction pathway, such as a MAP kinase pathway) in the same cells, and (4) express all the components of a pathway that synthesizes a small molecule (e.g., polyketide synthetase). In addition, the capacity to co-transfect allows the creation of supports with combinatorial combinations of different target biopolymers (e.g., co-expressed plasmids). This capacity is particularly useful for implementing mammalian two-hybrid assays in which plasmids encoding bait and prey proteins are co-transfected into the same cells by spotting them in one feature of a support.

Any suitable support can be used in the practice of the invention. For example, a solid support can be used and nucleic acid molecule containing mixtures may be applied to the surface of this support. As examples, the support can be glass, plastics (such as polytetrafluoroethylene, polyvinylidenedifluoride, polystyrene, polycarbonate, polypropylene), silicon, metal, (such as gold), membranes (such as nitrocellulose, methylcellulose, PTFE or cellulose), paper, biomaterials (such as protein, gelatin, agar), tissues (such as skin, endothelial tissue, bone, cartilage), minerals (such as hydroxylapatite, graphite). Additional compounds may be added to the base material of the support to provide functionality. For example, scintillants can be added to a polystyrene substrate to allow Scintillation Proximity Assays to be performed.

The support may be a porous solid support or non-porous solid support. The support can have a surface with concave or convex regions, patterns of hydrophobic or hydrophilic regions, diffraction gratings, channels or other features. The scale of these features can range from the meter to the nanometer scale. For example, the scale can be on the micron scale for microfluidic channels or other MEMS features or on the nanometer scale for nanotubes or buckyballs. The surface can be planar, planar with raised or sunken features, spherical (e.g. optically encoded beads), fibers (e.g. fiber optic bundles), tubular (both interior or exterior), a 3-dimensional network (such as interlinking rods, tubes, spheres) or other shapes. The surface can be part of an integrated system. For instance, the surface can be the bottom of a microtitre dish, a culture dish, a culture chamber. Other components, such as lenses, gratings, and electrodes, can be integrated with the surface. In general, the material of the support and geometry of the array will be selected based on criteria that it be useful for automation of array formation, culturing and/or detection of cellular phenotype.

In still other embodiments, the support is a microsphere (bead), especially a FACS sortable bead. In some instances, each bead is an individual feature (e.g., having a homogenous population of target sequences and distinct from most other beads in the mixture) and one or more tags which can be used to the identify any given bead and therefore the target sequence it displays. The identity of any given target sequence that can induce a FACS-detectable change in cells that adhere to the beads can be readily determined from the tag(s) associate with the bead. For example, the tag can be an electrophoric tagging molecules that are used as a binary code (Ohlmeyer et al. (1993) PNAS 90:10922-10926). Exemplary tags include haloaromatic alkyl ethers that are detectable as their trimethylsilyl ethers at less than femtomolar levels by electron capture gas chromatography (ECGC). Variations in the length of the alkyl chain, as well as the nature and position of the aromatic halide substituents, permit the synthesis of at least 40 such tags, which in principle can encode 240 (e.g., upwards of 1012) different molecules.

In addition, the surface can be coated with, for example, a cationic moiety. The cationic moiety can be any positively charged species capable of electrostatically binding to negatively charged target biopolymers. Exemplary cationic moieties for use in the carrier include polycations, such as polylysine (e.g., poly-L-lysine), polyarginine, polyornithine, spermine, basic proteins such as histones (Chen et al. (1994) FEBS Letters 338:167-169), avidin, protamines (see e.g., Wagner et al. (1990) PNAS 87:3410-3414), modified albumin (i.e., N-acylurea albumin) (see e.g., Huckett et al. (1990) Chemical Pharmacology 40:253-263), and polyamidoamine cascade polymers (see e.g., Haensler et al. (1993) Bioconjugate Chem. 4:372-379). One particularly useful polycation is polylysine (e.g., ranging from 3,800 to 60,000 daltons). Alternatively, the surface itself can be positively charged (such as gamma amino propyl silane or other alkyl silanes).

The surface may also be coated with molecules for additional functions. For instance, these molecules can be capture reagents such as antibodies, biotin, avidin, Ni-NTA to bind epitopes, avidin, biotinylted molecules, or 6-His tagged molecules. Alternatively, the molecules can be culture reagents such as extracellular matrix, fetal calf serum, and collagen.

V. Deposition Agents

In many instances, nucleic acid molecules used in the practice of the invention may be mixed with one or more (e.g., one, two, three, four, five, six, seven, eight, nine, ten, etc.) deposition agents. Deposition agents are compounds which may serve one or more of the following purposes: (1) add bulk to the composition which contains the nucleic acid molecules, (2) enhance the ability of nucleic acid molecules to adhere to a support, (3) enhance the ability of cells to adhere to a support, or (4) enhance the ability of cells to take up nucleic acid molecules (e.g., transfection reagents).

Any number of compounds may be used as deposition agents. Examples of such compounds include nucleic acids (e.g., RNA, DNA, etc.), proteins (e.g., bovine serum albumin, etc.), lipids, carbohydates (e.g., sugars, starches, gums, etc.). In particular instances, a deposition agent is not a protein (e.g., gelatin) and/or a nucleic acid.

In particular instances, a deposition agent may not be present in methods and compositions of the invention. For example, when cells are contacted with a support which contain viral particles and/or viruses, in many instances, the viral particles and/or viruses will contain all components necessary for entry of those particles and/or viruses into cells. In such instances, deposition agents may be omitted.

More specific examples of deposition agents include compositions which contain dextran, chitosan, karaya gum, polyacrylamide, xanthan gum, guar gum, acacia gum, pectin, starch, starch derivatives, vinyl acetate copolymer, polyvinyl pyrrolidone, polyethylene oxide, algin, derivatives of algin, polyacrylate, polymaleic acid, polymaleic anhydride, agarose, polyurethane, polyurea, gum acacia, locust bean gum, modified guar gum, maltodextrin, carboxymethyl cellulose, carboxypropyl cellulose, polyvinyl alcohol, poly AMPS, or a mixture thereof.

Thus, deposition agents include compositions which contain natural polymer, synthetic polymer, hydrophilic polymers, hydrophobic polymers, and hydrocolloidal polymers.

Gums have a number of physical properties that are advantageous in the preparation and use of transfected cell arrays: they act as emulsifiers and can thereby stabilize the lipid-target biopolymer complex (e.g., a lipid-DNA complex) in solution prior to spotting; they readily form gels at high concentrations, which can slow the drying of spotted transfection complex and thereby improve spot morphology and uniformity; they are extremely hydrophilic, which can prevent desiccation—and degradation—of reverse transfection spots; they are viscous, which can slow the dissolution and dispersion of the transfection complex during cell culture and thereby improve transfection efficiency; and they are exogenous, non-toxic and non-nutritive, and so should not adversely effect cell growth.

Proteins such as fibronectin and gelatin obtained from commercial sources can be expensive, unstable in aqueous solutions, and of variable quality. If the specific cell adhesion properties of proteins such as fibronectin do not play a role in transfection, then proteins commonly used in in vitro experiments (e.g., albumin, immunoglobulin) may provide an inexpensive, reliable alternative. Along these lines, we have found that bovine serum albumin works well in reverse transfection methods.

Another approach to reverse transfection is viral arrays. We have successfully prepared arrays of lentivirus encoding for green fluorescent protein and used these arrays to infect cells. Infection occurs in the absence of transfection reagent and is not effected by the addition of proteins to the spotting solution. However, the addition of guar gum and 100 mM sucrose proved beneficial (i.e., increased transfection efficiency).

Deposition agents may be used in the practice of the invention alone or in conjunction with other reagents (e.g., a transfection reagent). For example, when plasmid DNA is spotted on a solid support (e.g., a glass microscope slide), the DNA may be contained in a composition along with a transfection reagent such as Lipofectamine™ 2000 and guar gum.

Further, the ratio of the nucleic acid molecule concentration to the total deposition agent concentration in terms of weight to volume (μg/ml) may vary considerably. For example, the ratio of nucleic acid molecules to deposition agent may be 5:1, 10:1, 20:1, 25:1, 30:1, 35:1, 40:1, 50:1, 60:1, 80:1, etc. Additionally, the ratio of nucleic acid molecules to deposition agent may be in the ranges of 5:1 to 80:1, 5:1 to 40:1, 5:1 to 20:1, 5:1 to 10:1, 10:1 to 80:1, 10:1 to 60:1, 10:1 to 50:1, 10:1 to 40:1, 15:1 to 80:1, 20:1 to 40:1, etc.

Further, when a deposition agent is used in the practice of the invention in conjunction with another reagent (e.g., a transfection reagent), the deposition agent and the other reagent may be present in equal amounts or in different amounts. For example, when a transfection reagent is present in conjunction with a gum, the transfection reagent will typically be present in a concentration which is different than that of the gum contained in the deposition agent.

Transfection reagents which may be used in methods and compositions of the invention will typically include cationic and anionic lipids. Specific examples of transfection reagents include Lipofectin™ (Invitrogen Corp., Carlsbad, Calif., cat. no. 18292-037), Lipofectamine™ 2000 (Invitrogen Corp., Carlsbad, Calif., cat. no. 11668-019), Lipofectamine™ (Invitrogen Corp., Carlsbad, Calif., cat. no. 18324-020), Cellfectin™ (Invitrogen Corp., Carlsbad, Calif., cat. no. 10362-010), Optifect™ (Invitrogen Corp., Carlsbad, Calif., cat. no. 12579-017), TransFectin™ (Bio-Rad Laboratories, Inc., Hercules, Calif. 94547, cat. no. 170-3350), siLentFect™ (Bio-Rad Laboratories, Inc., Hercules, Calif. 94547, cat. no. 170-3360), SureFECTOR™ (B-Bridge International, Sunnyvale, Calif. 94085, cat. no. EM-101-001), UniFECTOR™ (B-Bridge International, Sunnyvale, Calif. 94085, cat. no. EM-101-002), Insect GeneJuice® (Novagen, Madison, Wis. 53719, cat. no. 71259-3), LipoTAXI® (Stratagene, La Jolla, Calif. 92037, cat. no. 204110), FuGene™ 6 (Roche Diagnostics, Basel, Switzerland), X tremeGENE Q2 (Roche Diagnostics, Basel, Switzerland), X tremeGENE siRNA (Roche Diagnostics, Basel, Switzerland), Effectene (Qiagen Inc, Valencia, Calif., cat. no. 301425), Superfect (Qiagen Inc, Valencia, Calif., cat. no. 301305), Polyfect (Qiagen Inc, Valencia, Calif., cat. no. 301105), TransMessenger (Qiagen Inc, Valencia, Calif., cat. no. 301525), TransIT-LT1 (Mirus, Madison, Wis. 53719, cat. no. MIR 2304), TransIT-LT2 (Mirus, Madison, Wis. 53719, cat. no. MIR 2404), TransIT-TKO (Mirus, Madison, Wis. 53719, cat. no. MIR 2154), and JetPEI (Avanti Polar Lipids, Inc. see, e.g., cat. no. 101-05N). Additional transfection reagents which may be used in the practice of the invention are set out in Appendix 1.

Any number of gums may be used as deposition agents in the practice of the invention. Examples of suitable gums include xanthan gum, guar gum, and locust bean gum. Additional suitable gums may be found at http address www.texturant-systems.com/texturant/html/e/products/prodover.htm.

One specific example of a gum which may be used in the practice of the invention is guar gum. The main chain of guar gum consists of (1-4) linked beta-D mannose residues and the side chain of (1-6) linked alpha-D galactose. Guar gum thus has an overall ratio of mannose to galactose of around 2:1. Further, the galactose substituents are regularly distributed along the mannose chain.

Another specific example of a gum which may be used in the practice of the invention is xanthan gum. Xanthan gum is an hetero-polysaccharide of high molecular weight. Its main chain is constituted of glucose units. The xanthan gum side chain is a trisaccharide, consisting of alpha-D-mannose which contains an acetyl group, of beta-D-glucuronic acid, and of a terminal beta-D-mannose unit linked with a pyruvate group.

Another class of compounds which may be used in the practice of the invention are pectins. Pectin molecules are basically chains of galacturonic acid units. The regular structure is interrupted by the presence of a methylpentose, L-rhamnose, which causes deviations called “pectic elbows”. The L-rhamnose is linked by carbons 1 and 2.

A certain proportion of these galacturonic acids are typically in the methyl ester form. The percentage of the galacturonic acids which are esterified is generally called the degree of esterification (DE) or degree of methoxylation (DM).

High methoxyl (HM) Pectins are normally referred to as those with a DE above 50, while low methoxyl (LM) Pectins have a DE of less than 50. LM Pectins can be acid- or alkali-treated. Further, LM Pectins can be either amidated (LMA) or non-amidated (LMNA). Any such pectins may be used in methods and compositions of the invention.

Another class of compounds which may be used in the practice of the invention are the carrageenans. Many red seaweeds contain polysaccharides made up of sulfated galactose units. These polysacchrides are often referred to as carrageenans. All the galactose units in carrageenans are typically in the D form. Further, the sulfate content in carrageenans is generally between 15 and 40%.

Another class of compounds which may be used in the practice of the invention are the locust bean gums. Many leguminous plant seeds contain galactomannans with similar structure. Those extracted from the locust bean are the most frequently used. The main chain consists of (1-4) linked beta-D mannose residues and the side chain (1-6) linked alpha-D galactose. The galactose sugars are not evenly distributed along the chain but tend to be clustered together in blocks. The chains have an irregular structure with alternating “smooth” and substituted zones.

Another deposition agent which may be used in practice of the invention is scleroglucan. Scleroglucan is a water soluble natural polymer produced by fermentation of the filamentous fungi Sclerotium rofsii (see U.S. Pat. No. 4,647,312, the entire disclosure of which is incorporated herein by reference).

VI. Proteins

As indicated above, features may contain target biopolymers, such as nucleic acid molecules, proteins, and carbohydrates, as well as other compounds which one desires to introduce into cells.

When the target biopolymer is a protein, the protein may have any number of characteristics. Examples of characteristics of proteins which may vary include size, amino acid sequence, and functional activities.

In terms of size, features may contain proteins of small size (e.g., from about two amino acids to about twenty amino acids), commonly referred to as “peptides”, of intermediate size (e.g., from about twenty amino acids to about 200 amino acids), or large size (e.g., from about 200 amino acids to about 20,000 amino acids). Exemplary sizes of proteins which may be present in features are from about 2 to about 20 amino acids, from about 10 to about 30 amino acids, from about 20 to about 40 amino acids, from about 30 to about 80 amino acids, from about 20 to about 100 amino acids, from about 50 to about 100 amino acids, from about 50 to about 200 amino acids, from about 50 to about 500 amino acids, from about 2 to about 400 amino acids, from about 100 to about 1,000 amino acids, or from about 200 to about 2,000 amino acids.

Further, proteins contained in features may be one chain or multi-chain (i.e., multimeric). When protein multi-chain proteins are included within features, these features, in essence, contain more than one protein (i.e., the individual proteins which form the multi-chain entity).

Proteins used in the practice of the invention, whether contained in or encoded by nucleic acid molecules of features, may be fusion proteins. For example, a domain of one protein may be connected to a domain of another protein. Such domains include SH1 domains, SH2 domains, zinc finger domains, domains which bind to cell surface proteins, and transmembrane domains.

Proteins used in various aspects of the invention may also domains which allow for translocation of proteins across cell membranes. One example of such a domain is Domain II of Pseudomonas exotoxin (see, e.g., Pastan et al., U.S. Pat. No. 5,328,984, the entire disclosure of which is incorporated herein by reference). Examples of proteins with membrane translocation activity include, but are not limited to, the plant and bacterial protein toxins ricin, abrin, modeccin, diphtheria toxin, cholera toxin, anthrax toxin. Examples of proteins that are not toxins but also have membrane translocation activity, include the TAT protein of human immunodeficiency virus and the protein VP22, which is the product of the UL49 gene of herpes simplex virus type 1.

The invention thus includes the use of features which contain proteins capable of translocation across cell membranes. Thus, the invention includes supports with features which contain proteins capable of translocation across cell membranes, as well as methods employing such supports.

VII. Cells

Suitable host cells for generating the subject assay include prokaryotes, yeast, or higher eukaryotic cells, including plant and animal cells, especially mammalian cells. Prokaryotes include gram negative or gram positive organisms.

In certain embodiments, methods of the invention may be carried out using cells derived from higher eukaryotes (e.g., metazoans), such as mammalian cells (e.g., primate cells such as human cells). Species of mammalian cells include canine, feline, bovine, porcine, mouse and rat cells.

Animal cells used in methods of the invention can be hematopoietic cells, neuronal cells, pancreatic cells, hepatic cells, chondrocytes, osteocytes, or myocytes.

The cells can be fully differentiated cells or progenitor/stem cells. Moreover, the cells may be derived from normal or diseased tissue, from differentiated or undifferentiated cells, from embryonic or adult tissue. The cells may be dispersed in culture, or can be tissues samples containing multiple cells which retain some of the microarchitecture of the organ.

In certain embodiments, supports of the invention may be used to transfect a cell that can be co-cultured with a target cell. Under particular conditions, biologically active proteins may be secreted by the cells expressing genes from features, which will then diffuse to neighboring target cells and induce a particular biological response (e.g., proliferation or differentiation, activation of a signal transduction pathway which is directly detected by other phenotypic criteria, etc.). Likewise, antagonists of a given factor can be selected in similar fashion by the ability of the cell producing a functional antagonist to protect neighboring cells from the effect of exogenous factor added to the culture media. The host and target cells can be in direct contact, or separated by, for example, a cell culture insert (see, e.g., Collaborative Biomedical Products, a division of Becton Dickinson, Bedford, Mass., cat. no. 40446).

The choice of appropriate host cell will also be influenced by such factors as the choice of detection signal and/or target biopolymer and the cell type that one wishes to study. For instance, reporter constructs can provide a selectable or screenable trait upon gain-of-function or loss-of-function induced by a target nucleic acid. The reporter gene may be an unmodified gene already in the host cell pathway, or it may be a heterologous gene (e.g., a “reporter gene construct”). In other embodiments, second messenger generation can be measured directly in a detection step, such as mobilization of intracellular calcium or phospholipid metabolism, in which case the host cell should have an appropriate starting phenotype for activation of such pathways.

Host cells may be plated (placed) onto feature bearing support in sufficient density and under appropriate conditions for introduction/entry of the target biopolymers into the cells. In some instances, the host cells (in an appropriate medium) may be plated on the array at high density (e.g., on the order of 0.5-1×10⁵/cm²), in order to increase the likelihood that transfection will occur or to increase the number of cells which are transfected with target biopolymers from each feature. For example, the density of cells can be from about 0.3×10⁵/cm² to about 3×10⁵/cm², and in specific embodiments, is from about 0.5×10⁵/cm² to about 2×10⁵/cm² and from about 0.5×10⁵/cm² to about 1×10⁵/cm². The appropriate conditions for introduction/entry of target biopolymers into cells will vary with factors such as the quantity of cells used, the transfection efficiency of the system, and the particular cells employed.

VIII. Business Methods

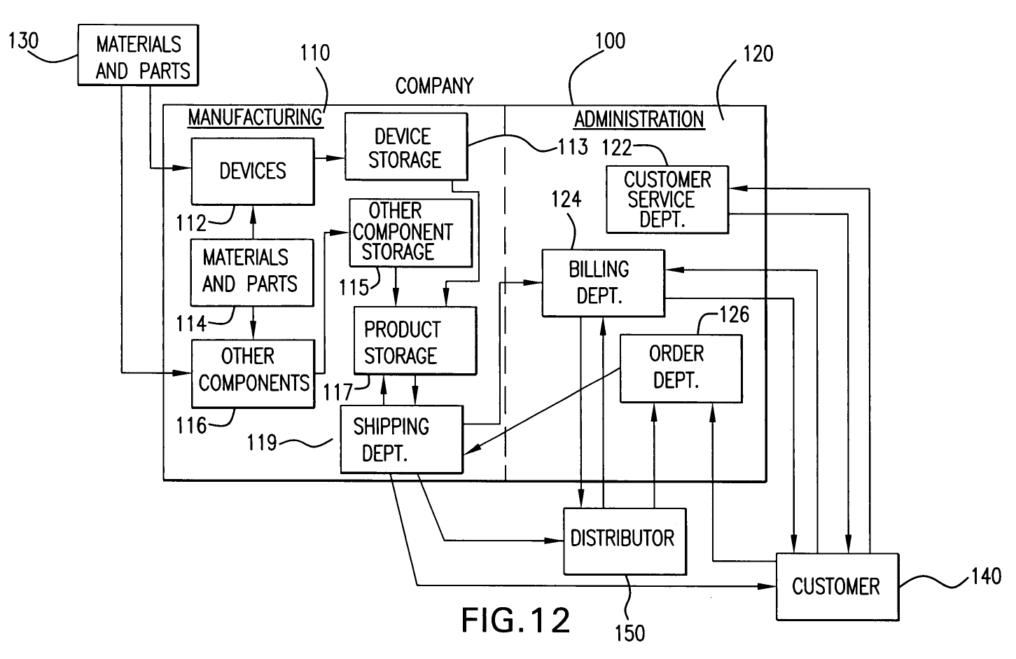

The present invention also provides a system and method of providing company products to a party outside of the company, for example, a system and method for providing a customer or a product distributor a product of the company such as a kit containing a support having one or more feature. FIG. 12 provides a schematic diagram of a product management system. In practice, the blocks in FIG. 12 can represent an intra-company organization, which can include departments in a single building or in different buildings, a computer program or suite of programs maintained by one or more computers, a group of employees, a computer I/O device such as a printer or fax machine, a third party entity or company that is otherwise unaffiliated with the company, or the like.

The product management system as shown in FIG. 12 is exemplified by company 100, which receives input in the form of an order from a party outside of the company, e.g., distributor 150 or customer 140, to order department 126, or in the form of materials and parts 130 from a party outside of the company; and provides output in the form of a product delivered from shipping department 119 to distributor 150 or customer 140. Company 100 system is organized to optimize receipt of orders and delivery of a products to a party outside of the company in a cost efficient manner, particularly instructions or a kit of the present invention, and to obtain payment for such product from the party.

With respect to methods of the present invention, the term “materials and parts” refers to items that are used to make a device, other component, or product, which generally is a device, other component, or product that company sells to a party outside of the company. As such, materials and parts include, for example, nucleic acid molecules, support, deposition agents, host cells, enzymes (e.g., polymerases), amino acids, culture media, buffers, paper, ink, reaction vessels, etc. In comparison, the term “devices”, “other components”, and “products” refer to items sold by the company. Devices are exemplified supports which contain one or more feature. Other components are exemplified by instructions, including instructions for preparing and using supports which contain features. Other components also can be items that may be included in a kit. As such, it will be recognized that an item useful as materials and parts as defined herein further can be considered an “other component”, which can be sold by the company. The term “products” refers to devices, other components, or combinations thereof, including combinations with additional materials and parts, that are sold or desired to be sold or otherwise provided by a company to one or more parties outside of the company. Products are exemplified herein by supports which contain features and kits (e.g., kits which can contain instructions according to the present invention).

Referring to FIG. 12, company 100 includes manufacturing 110 and administration 120. Devices 112 and other components 114 are produced in manufacturing 110, and can be stored separately therein such as in device storage 113 and other component storage 115, respectively, or can be further assembled and stored in product storage 117. Materials and parts 130 can be provided to company 100 from an outside source and/or materials and parts 114 can be prepared in company, and used to produce devices 112 and other components 116, which, in turn, can be assembled and sold as a product. Manufacturing 110 also includes shipping department 119, which, upon receiving input as to an order, can obtain products to be shipped from product storage 117 and forward the product to a party outside the company.

For purposes of the present invention, product storage 117 can store instructions, for example, for preparing and using supports with features, as well as combinations of such instructions and/or kits. Upon receiving input from order department 126, for example, that customer 140 has ordered such a kit and instructions, shipping department 119 can obtain from product storage 117 such kit for shipping, and can further obtain such instructions in a written form to include with the kit, and ship the kit and instructions to customer 140 (and providing input to billing department 124 that the product was shipped; or shipping department 119 can obtain from product storage 117 the kit for shipping, and can further provide the instructions to customer 140 in an electronic form, by accessing a database in company 100 that contains the instructions, and transmitting the instructions to customer 140 via the internet (not shown).

As further exemplified in FIG. 12, administration 120 includes order department 126, which receives input in the form of an order for a product from customer 140 or distributor 150. Order department 126 then provides output in the form of instructions to shipping department 119 to fill the order (i.e., to forward products as requested to customer 140 or distributor 150). Shipping department 119, in addition to filling the order, further provides input to billing department 124 in the form of confirmation of the products that have been shipped. Billing department 124 then can provide output in the form of a bill to customer 140 or distributor 150 as appropriate, and can further receive input that the bill has been paid, or, if no such input is received, can further provide output to customer 140 or distributor 150 that such payment may be delinquent. Additional optional component of company 100 include customer service department 122, which can receive input from customer 140 and can provide output in the form of feedback or information to customer 140. Furthermore, although not shown in FIG. 12, customer service 122 can receive input or provide output to any other component of company. For example, customer service department 122 can receive input from customer 140 indicating that an ordered product was not received, wherein customer service department 122 can provide output to shipping department 119 and/or order department 126 and/or billing department 124 regarding the missing product, thus providing a means to assure customer 140 satisfaction. Customer service department 122 also can receive input from customer 140 in the form of requested technical information, for example, for confirming that instructions of the invention can be applied to the particular need of customer 140, and can provide output to customer 140 in the form of a response to the requested technical information.

As such, the components of company 100 are suitably configured to communicate with each other to facilitate the transfer of materials and parts, devices, other components, products, and information within company 100, and company 100 is further suitably configured to receive input from or provide output to an outside party. For example, a physical path can be utilized to transfer products from product storage 117 to shipping department 119 upon receiving suitable input from order department 126. Order department 126, in comparison, can be linked electronically with other components within company 100, for example, by a communication network such as an intranet, and can be further configured to receive input, for example, from customer 140 by a telephone network, by mail or other carrier service, or via the internet. For electronic input and/or output, a direct electronic link such as a T1 line or a direct wireless connection also can be established, particularly within company 100 and, if desired, with distributor 150 or materials or parts 130 provider, or the like.

Although not illustrated, company 100 system one or more data collection systems, including, for example, a customer data collection system, which can be realized as a personal computer, a computer network, a personal digital assistant (PDA), an audio recording medium, a document in which written entries are made, any suitable device capable of receiving data, or any combination of the foregoing. Data collection systems can be used to gather data associated with a customer 140 or distributor 150, including, for example, a customer's shipping address and billing address, as well as more specific information such as the customer's ordering history and payment history, such data being useful, for example, to determine that a customer has made sufficient purchases to qualify for a discount on one or more future purchases.

Company 100 can utilize a number of software applications to provide components of company 100 with information or to provide a party outside of company access to one or more components of company 100, for example, access to order department 126 or customer service department 122. Such software applications can comprise a communication network such as the Internet, a local area network, or an intranet. For example, in an internet-based application, customer 140 can access a suitable web site and/or a web server that cooperates with order department 126 such that customer 140 can provide input in the form of an order to order department 126. In response, order department 126 can communicate with customer 140 to confirm that the order has been received, and can further communicate with shipping department 119, providing input that products such as a kit of the invention, which contains, for example, a support having one or more feature and instructions for use, should be shipped to customer 140. In this manner, the business of company 100 can proceed in an efficient manner.

In a networked arrangement, billing department 124 and shipping department 119, for example, can communicate with one another by way of respective computer systems. As used herein, the term “computer system” refers to general purpose computer systems such as network servers, laptop systems, desktop systems, handheld systems, personal digital assistants, computing kiosks, and the like. Similarly, in accordance with known techniques, distributor 150 can access a web site maintained by company 100 after establishing an online connection to the network, particularly to order department 126, and can provide input in the form of an order. If desired, a hard copy of an order placed with order department 126 can be printed from the web browser application resident at distributor 150.

The various software modules associated with the implementation of the present invention can be suitably loaded into the computer systems resident at company 100 and any party outside of company 100 as desired, or the software code can be stored on a computer-readable medium such as a floppy disk, magnetic tape, or an optical disk. In an online implementation, a server and web site maintained by company 100 can be configured to provide software downloads to remote users such as distributor 150, materials and parts 130, and the like. When implemented in software, the techniques of the present invention are carried out by code segments and instructions associated with the various process tasks described herein.

Accordingly, the present invention further includes methods for providing various aspects of a product (e.g., a kit and/or instructions of the invention), as well as information regarding various aspects of the invention, to parties such as the parties shown as customer 140 and distributor 150 in FIG. 12. Thus, methods for selling devices, products and methods of the invention to such parties are provided, as are methods related to those sales, including customer support, billing, product inventory management within the company, etc. Examples of such methods are shown in FIG. 12, including, for example, wherein materials and parts 130 can be acquired from a source outside of company 100 (e.g., a supplier) and used to prepare devices used in preparing a composition or practicing a method of the invention, for example, kits, which can be maintained as an inventory in product storage 117. It should be recognized that devices 112 can be sold directly to a customer and/or distributor (not shown), or can be combined with one or more other components 116, and sold to a customer and/or distributor as the combined product. The other components 116 can be obtained from a source outside of company 100 (materials and parts 130) or can be prepared within company 100 (materials and parts 114). As such, the term “product” is used generally herein to refer an item sent to a party outside of the company (a customer, a distributor, etc.) and includes items such as devices 112, which can be sent to a party alone or as a component of a kit or the like.

At the appropriate time, the product is removed from product storage 117, for example, by shipping department 119, and sent to a requesting party such as customer 140 or distributor 150. Typically, such shipping occurs in response to the party placing an order, which is then forwarded the within the organization as exemplified in FIG. 12, and results in the ordered product being sent to the party. Data regarding shipment of the product to the party is transmitted further within the organization, for example, from shipping department 119 to billing department 124, which, in turn, can transmit a bill to the party, either with the product, or at a time after the product has been sent. Further, a bill can be sent in instances where the party has not paid for the product shipped within a certain period of time (e.g., within 30 days, within 45 days, within 60 days, within 90 days, within 120 days, within from 30 days to 120 days, within from 45 days to 120 days, within from 60 days to 120 days, within from 90 days to 120 days, within from 30 days to 90 days, within from 30 days to 60 days, within from 30 days to 45 days, within from 60 days to 90 days, etc.). Typically, billing department 124 also is responsible for processing payment(s) made by the party. It will be recognized that variations from the exemplified method can be utilized; for example, customer service department 122 can receive an order from the party, and transmit the order to shipping department 119 (not shown), thus serving the functions exemplified in FIG. 12 by order department 126 and the customer service department 122.

Methods of the invention also include providing technical service to parties using a product, particularly a kit of the invention. While such a function can be performed by individuals involved in product research and development, inquiries related to technical service generally are handled, routed, and/or directed by an administrative department of the organization (e.g., customer service department 122). Often communications related to technical service (e.g., solving problems related to use of the product or individual components of the product) require a two way exchange of information, as exemplified by arrows indicating pathways of communication between customer 150 and customer service department 122.

As mentioned above, any number of variations of the process exemplified in FIG. 12 are possible and within the scope of the invention. Accordingly, the invention includes methods (e.g., business methods) that involve (1) the production of products; (2) receiving orders for these products; (3) sending the products to parties placing such orders; (4) sending bills to parties obliged to pay for products sent to such; and/or (5) receiving payment for products sent to parties. For example, methods are provided that comprise two or more of the following steps: (a) obtaining parts, materials, and/or components from a supplier; (b) preparing one or more first products (e.g., one or more supports with one or more features); (c) storing the one or more first products of step (b); (d) combining the one or more first products of step (b) with one or more other components to form one or more second products (e.g., a kit); (e) storing the one or more first products of step (b) or one or more second products of step (d); (f) obtaining an order a first product of step (b) or a second product of step (d); (g) shipping either the first product of step (b) or the second product of step (d) to the party that placed the order of step (f); (h) tracking data regarding to the amount of money owed by the party to which the product is shipped in step (g); (i) sending a bill to the party to which the product is shipped in step (g); (j) obtaining payment for the product shipped in step (g) (generally, but not necessarily, the payment is made by the party to which the product was shipped in step (g); and (k) exchanging technical information between the organization and a party in possession of a product shipped in step (d) (typically, the party to which the product was shipped in step (g)).

Nucleic acid molecules used in compositions and methods of the invention may be designed so that they do not replicate efficiently in commonly used cells types. An advantage of this is so that purchasers can not easily propagate nucleic acid molecules in commonly used cloning systems (e.g., Escherichia coli based systems, yeast based systems, etc.). Thus, for example, when arrays of the invention contain plasmids, these plasmids may contain one or more of the following: (1) an origin of replication which is not compatible with commonly used cloning systems (e.g., a non-E. coli origin, such as an origin of replication which functions only in Gram positive organisms or yeast such as the 2 micron plasmid origin or a yeast autonomously replicating sequence; a temperature sensitive origin of replication, such as the one described in U.S. Patent Publication No. 2003/0124555, the entire disclosure of which is incorporated herein by reference; the R6K-gamma origin of replication may be used which will allows for propagation in E. coli only when the pir gene is present; etc.); (2) a terminator sequence oriented in manner to prevent replication of the plasmid in E. coli which express tus (the tus/ter system is described in U.S. Patent Publication 2003/0176644, the entire disclosure of which is incorporated herein by reference); (3) a gene which expresses a product that is either toxic to particular cells or toxic to particular cells under particular conditions (e.g., one or more of the following genes: ccdB, sacB, rpsL, tetAR, pheS, thyA, lacY, gata-1, colicin E1, barnase, etc., described in U.S. Pat. No. 6,818,441; Jucovic et al., Proc. Natl. Acad. Sci (USA) 93:2343-2347 (1996); and Ryerat et al., Infection and Immunity 66:4011-4017 (1998), the entire disclosures of which is incorporated herein by reference); or (4) a transcriptional regulatory sequence (e.g., an inducible promoter, such as the pBAD promoter or a metalothionine promoter; a repressible promoter, such as a tet operator; etc.) operably connected to (a) a positive selectable marker (e.g., a kanamycin resistance gene, an ampicillin resistance gene, a hygromycin resistance gene, a Zeocin™ resistance gene, a Blasticidin™ resistance gene, etc.) or (b) a negative selectable marker (e.g., a gene of (3) set out above). Thus, the invention relates to “copy control” systems. Of course, these copy control component(s) may be used with nucleic acid molecules other than plasmids. The choice of copy control component(s) included used will often be determined, for example, by the cloning system(s) which may be employed by purchasers to propagate nucleic acid molecules of arrays of the invention.

The present invention also provides a system and method for providing information as to availability of a product (e.g., a device product, a kit product, and the like) to parties having potential interest in the availability of the kit product. Such a method of the invention, which encompasses a method of advertising to the general or a specified public, the availability of the product, particularly a product comprising instructions and/or a kit of the present invention, can be performed, for example, by transmitting product description data to an output source, for example, an advertiser; further transmitting to the output source instructions to publish the product information data in media accessible to the potential interested parties; and detecting publication of the data in the media, thereby providing information as to availability of the product to parties having potential interest in the availability of the product.

Accordingly, the present invention provides methods for advertising and/or marketing devices, products, and/or methods of the invention, such methods providing the advantage of inducing and/or increasing the sales of such devices, products, and/or methods. For example, advertising and/or marketing methods of the invention include those in which technical specifications and/or descriptions of devices and/or products; methods of using the devices and/or products; and/or instructions for practicing the methods and/or using the devices and/or products are presented to potential interested parties, particularly potential purchasers of the product such as customers, distributors, and the like. In particular embodiments, the advertising and/or marketing methods involve presenting such information in a tangible form or in an intangible to the potential interested parties. As disclosed herein and well known in the art, the term “intangible form” means a form that cannot be physically handled and includes, for example, electronic media (e.g., e-mail, internet web pages, etc.), broadcasts (e.g., television, radio, etc.), and direct contacts (e.g., telephone calls between individuals, between automated machines and individuals, between machines, etc.); whereas the term “tangible form” means a form that can be physically handled.

The invention further provides methods associated with the design of custom products. These methods include, for example, (1) the taking an order from a customer for supports with specific target biopolymers in one or more features, (2) preparation of one or more support which contains the particular specific target biopolymers in one or more features, (3) and providing (e.g., shipping) the support of (b) to the customer. Additionally, in particular embodiments, the customer may be billed for the support with the bill either being sent to the customer along with the support or sent separately.

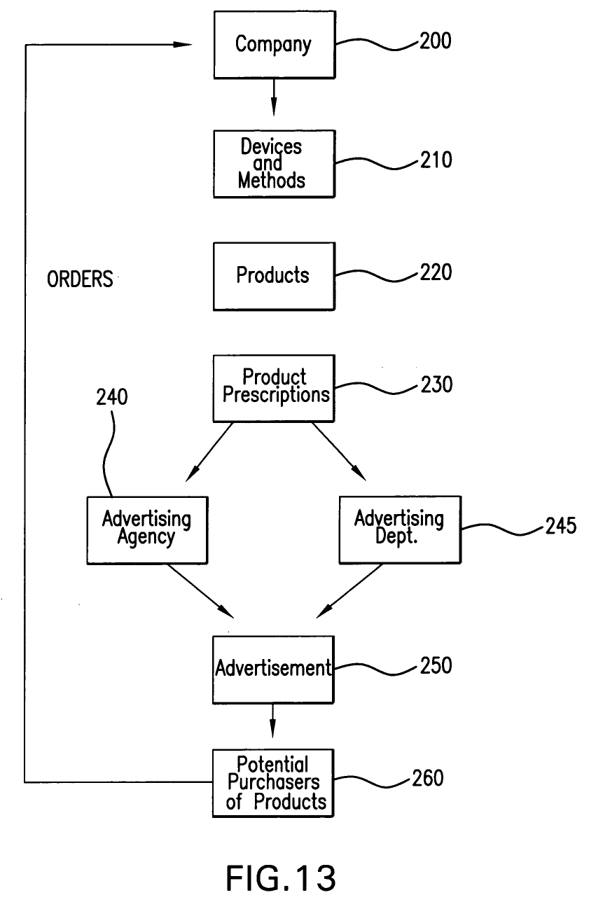

FIG. 13 provides a schematic diagram of an information providing management system as encompassed within the present invention. In practice, the blocks in FIG. 13 can represent an intra-company organization, which can include departments in a single building or in different buildings, a computer program or suite of programs maintained by one or more computers, a group of employees, a computer I/O device such as a printer or fax machine, a third party entity or company that is otherwise unaffiliated with the company, or the like.

The information providing management system as shown in FIG. 13 is exemplified by company 200, which makes, purchases, or otherwise makes available devices and methods 210 that alone, or in combination, provide products 220, for example, instructions, devices and/or kits of the present invention, that company 200 wishes to sell to interested parties. To this end, product descriptions 230 are made, providing information that would lead potential users to believe that products 220 can be useful to user. In order to effect transfer of product descriptions 230 to the potential users, product descriptions 230 is provided to advertising agency 240, which can be an entity separate from company 200, or to advertising department 260, which can be an entity related to company 200, for example, a subsidiary. Based on the product descriptions 230, advertisement 250 is generated and is provided to media accessible to potential purchasers of products 260, whom may then contact company 200 to purchase products 220.

By way of example, product descriptions 230 can be in a tangible form such as written descriptions, which can be delivered (e.g., mailed, couriered, etc) to advertising agency 240 and/or advertising department 250, or can be in an intangible form such as entered into and stored in a database (e.g., on a computer, in an electronic media, etc.) and transmitted to advertising agency 240 and/or advertising department 250 over a telephone line, T1 line, wireless network, or the like. Similarly, advertisement 250 can be a tangible or intangible form such that it conveniently and effectively can be provided to potential parties of interest (e.g., potential purchasers of product 260). For example, advertisement 250 can be provided in printed form as flyers (e.g., at a meeting or other congregation of potential interested parties) or as printed pages (or portions thereof) in magazines known to be read by the potential interested parties (e.g., trade magazines, journals, newspapers, etc.). In addition, or alternatively, advertisement 250 can be provided in the form of directed mailing of computer media containing the advertisement (e.g., CDs, DVDs, floppy discs, etc.) or of e mail (i.e., mail or e-mail that is sent only to selected parties, for example, parties known to members of an organization that includes or is likely to include potential users of products 220); of web pages (e.g., on a website provided by company 200, or having links to the company 200 website); or of pop-up or pop-under ads on web pages known to be visited by potential purchaser of products 260, and the like. Potential purchasers of products 260, upon being apprised of the availability of the products 220, for example, the kits of the present invention, then can contact company 200 and, if so desired, can order said products 220 for company 200 (see FIG. 12).

IX. Kits and Instructions

The invention also provides kits. In various aspects, a kit of the invention may contain one or more (e.g., one, two, three, four, five, six, seven, etc.) of the following components: (1) one or more sets of instructions, including, for example, instructions for performing methods of the invention or for preparing and/or using compositions of the invention; (2) one or more cells, including, for example, one or more mammalian cells; (3) one or more support (either containing or not containing one or more feature); (4) one or more deposition agents; (5) one or more target biopolymers or other compound, as described herein; (6) one or more container containing water (e.g., distilled water) or other aqueous or liquid material; (6) one or more containers containing one or more buffers, which can be buffers in dry, powder form or reconstituted in a liquid such as water, including in a concentrated form such as 2×, 3×, 4×, 5×, etc.); (7) one or more culture medium; and/or (8) one or more containers containing one or more salts (e.g., sodium chloride, potassium chloride, magnesium chloride, which can be in a dry, powder form or reconstituted in a liquid such as water).

As suggested above, kits of the invention may contain one or more support either with or without features. Thus, supports may be “printed” before being provided to customers or may be provided to customers either without features (i.e., may be blank) or with printed with some features with additional features being added by the customer.

When a blank support is provided to a customer, feature content may also be provided. Thus, target biopolymers may be provided to the customers. This content may be provided alone or in conjunction with a support. Thus, the invention separately includes feature content. Often feature content will be provided along with one or more deposition agent.

Thus, in particular embodiments, the invention provides products which contain contents for one or more feature and one or more deposition agent (e.g., a deposition agent containing one or more gum). When a product of this type is provided to a customer, that customer may obtain a support from another source and may then add features to the support.

A kit of the invention can include an instruction set, or the instructions can be provided independently of a kit. Such instructions may provide information regarding how to make or use one or more of the following items: (1) a support with one or more feature; or (2) one or more array of transfected cells.

Instructions can be provided in a kit, for example, written on paper or in a computer readable form provided with the kit, or can be made accessible to a user via the internet, for example, on the world wide web at a URL (uniform resources link; i.e., “address”) specified by the provider of the kit or an agent of the provider. Such instructions direct a user of the kit or other party of particular tasks to be performed or of particular ways for performing a task. In one aspect, the instructions instruct a user of how to perform methods of the invention. In a specific aspect, the instructions can, for example, instruct a user of a kit as to reaction conditions for knocking-down gene expression, including, for example, buffers, temperature, and/or time periods of incubations for using nucleic acid molecules described herein. Instructions of the invention can be in a tangible form, for example, printed or otherwise imprinted on paper, or in an intangible form, for example, present on an internet web page at a defined and accessible URL. Thus, the invention includes instructions for performing methods of the invention and/or for preparing compositions of the invention. While the instructions themselves are one aspect of the invention, the invention also includes the instructions in tangible form. Thus, the invention includes computer media (e.g., hard disks, floppy disks, CDs, etc.) and sheets of paper (e.g., a single sheet of paper, a booklet, etc.) which contain the instructions.

It will be recognized that a full text of instructions for performing a method of the invention or, where the instructions are included with a kit, for using the kit, need not be provided. One example of a situation in which a kit of the invention, for example, would not contain such full length instructions is where the provided directions inform a user of the kits where to obtain instructions for practicing methods for which the kit can be used. Thus, instructions for performing methods of the invention can be obtained from internet web pages, separately sold or distributed manuals or other product literature, etc. The invention thus includes kits that direct a kit user to one or more locations where instructions not directly packaged and/or distributed with the kits can be found. Such instructions can be in any form including, but not limited to, electronic or printed forms.

The invention is further illustrated by the following examples, which should not be construed as limiting.

### EXAMPLES

Transfected cell microarray preparation and imaging. Samples of lipid-DNA complex to be used in producing transfection arrays for overexpression experiments were prepared by combining a pre-mixture of lipofection reagent and plasmid DNA with a pre-mixture of protein and gum, and manually arraying the resulting solution on a microscope slide. Such a solution was prepared by first diluting the plasmid in water (1 μl of 1 mg/ml aqueous stock plus 7.4 μl water) and then adding the lipofection reagent (6 μl of LIPOFECTAMINE™ 2000, 1 mg/ml). After a 20 minute incubation at room temperature, sucrose (2.1 μl of 1.6 M aqueous stock) was added to the lipid-DNA premixture. An aliquot of this pre-mixture was then added to an equal volume of the protein-gum premixture.

The protein-gum premixture was prepared as follows: 5 μl of 0.2 mg/ml BSA stock in water (prepared from a 50 mg/ml aqueous stock; Invitrogen Corp., Carlsbad, Calif., cat. no. 15561-020) plus 5 μl of 0.2 mg/ml aqueous stock of guar gum (a 1 mg/ml guar stock was prepared as follows: 10 mg of solid guar (Sigma-Aldrich, St. Louis, Mo., cat. no. G-4129) was slowly added to 10 ml water in a 50 ml conical tube under vigorous vortexing. The resulting mixture was kept at 4° C. overnight to allow for hydration of the gum particles, divided into ten 1 ml aliquots, and centrifuged in 1.5 ml snap-top tubes for 10 minutes at max speed in a microcentrifuge; the top 800 μl of the resulting supernatants were transferred by pipet, combined in a clean 15 ml conical, and stored at 4° C. prior to use).

Samples prepared as described above were immediately arrayed on microscope slides and the arrays were dried and stored at room temperature prior to use. Arrays were prepared manually with the aid of an electronic pipetor (a Rainin EDP3-LTS 10): 10 μl of sample was drawn into the pipet tip, 0.15 μl was dispensed to the end of the tip and transferred to the slide by touching the tip to the slide surface. A line of tick marks along the edge of the slide can be scribed with a carbide pen and used to plan the spotting of the array. A typical array prepared this way has 18 rows of spots with 5-8 replicate spots per row. The spot diameter ranges from 1.0 to 1.5 mm with as little as a 2 mm spot center to spot center spacing. The arrays were allowed to dry at room temperature overnight in a heat-sealed foil pouch that contained several desiccant packets.

Seeding of the arrays with cells was done by slowly lowering the slide array-side up into a 10 cm petri dish containing 20 ml of cells at a density of 6.2×10⁵/ml. Direct contact with the array either through touching it to a solid surface or pouring liquid over it was avoided to preserve the integrity of the spots. The slide was kept in the dish at 37° C. for 20-48 hours.

Arrays were imaged using an inverted microscope fitted with a CCD camera and operated with image acquisition/analysis software. Spot morphology was assessed prior to cell seeding by viewing the array under phase contrast microscopy (FIG. 3). Cell transfection was measured as GFP fluorescence by epifluorescence microscopy (FIGS. 4-6).

The entirety of each patent, patent application, publication and document referenced herein hereby is incorporated by reference, including all tables, drawings, and figures. All patents and publications are herein incorporated by reference to the same extent as if each was specifically and individually indicated to be incorporated by reference. Citation of the above patents, patent applications, publications and documents is not an admission that any of the foregoing is pertinent prior art, nor does it constitute any admission as to the contents or date of these publications or documents. Patents and publications mentioned herein are indicative of the skill levels of those of ordinary skill in the art to which the invention pertains.

Modifications may be made to the foregoing without departing from the scope, spirit and basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. One skilled in the art readily appreciates that the present invention is well adapted to carry out the objects and obtain the ends and advantages mentioned, as well as those inherent therein. The examples provided herein are representative of specific embodiments, are exemplary, and are not intended as limitations on the scope of the invention.

The invention illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

APPENDIX 1
NON-LIMITING EXAMPLES OF TRANSFECTION REAGENTS

| TRANSFECTION | | | |
|---|---|---|---|
| AGENT | DESCRIPTION | PATENTS AND/OR CITATIONS | AVAILABLE FROM |
| BMOP | N-(2-bromoethyl)-N,N-dimethyl- | | |
| | 2,3-bis(9-octadecenyloxy)- | | |
| | propanaminimun bromide) | | |
| BMOP:DOPE | 1:1 (wt/wt) formulation of N-(2- | Poult Sci 1997 Jun; 76(6): 882-6. | |
| | bromoethyl)-N,N-dimethyl-2,3- | Transfection of avian LMH-2A hepatoma | |
| | bis(9-octadecenyloxy)- | cells with cationic lipids. | |
| | propanaminimun bromide) (BMOP) and | Walzem R L, Hickman M A, German J B, | |
| | DOPE | Hansen R J. | |
| Cationic | Cationic polysaccharides | Published U.S. patent application | |
| polysaccharides | | 2002/0146826 | |
| CellFECTIN ® | 1:1.5 (M/M) formulation of N,NI, | U.S. Pat. Nos. 5,674,908, 5,834,439 | Invitrogen (LTI) |
| | NII,NIII-tetramethyl-N,NI,NII, | and 6,110,916 | |
| | NIII-tetrapalmitylspermine (TM- | | |
| | TPS) and dioleoyl | | |
| | phosphatidylethanolamine (DOPE) | | |
| CLONfectin ™ | N-t-butyl-N′-tetradecyl-3- | Ruysschaert, J. M., et al. (1994) | BD Biosciences Clontech |
| | tetradecyl-aminopropion-amidine | Biochem. Biophys. | |
| | | Res. Comm. 203: 1622-1628 | |
| CTAB:DOPE | formulation of cetyltrimethyl- | | |
| | ammonium bromide (CATB) and | | |
| | dioleoylphosphatidylethanolamine | | |
| | (DOPE) | | |
| Cytofectene | proprietary cationic lipid and DOPE | | Bio-Rad Laboratories |
| Cytofectin GSV | 2:1 (M/M) formulation of cytofectin | | (*Cytofectin GS |
| | GS* and dioleoyl phosphatidyl- | | corresponds to Gilead |
| | ethanolamine (DOPE) | | Sciences' GS 3815) |
| DC-Cholesterol (DC- | 3,β-N,(N′,N′- | | |
| Choi) | dimethylaminoethane)- | | |
| | carbamoyl]cholesterol | | |
| DC-Chol:DOPE | formulation of 3,β-N,(N′,N′- | Gao et al., Biochim. Biophys. | |
| | dimethylaminoethane)- | Res. Comm. 179: 280-285 (1991) | |
| | carbamoyl]cholesterol (DC-Chol) and | | |
| | dioleoyl phosphatidylethanolamine | | |
| | (DOPE) | | |
| DC-6-14 | O,O′-Ditetradecanoyl-N-(alpha- | Hum Gene Ther 1999 Apr 10; 10(6): | |
| | trimethylammonioacetyl)diethanolamine | 947-55. Development of novel | |
| | chloride | cationic liposomes for efficient | |
| | | gene transfer into peritoneal | |
| | | disseminated tumor. Kikuchi A, | |
| | | Aoki Y, Sugaya S, Serikawa T, | |
| | | Takakuwa K, Tanaka K, Suzuki N, | |
| | | Kikuchi H. | |
| DCPE | Dicaproylphosphtidylethanol-amine | | |
| DDPES | Dipalmitoylphosphatidyl- | Behr et al. 1989. Efficient gene | |
| | ethanolamine 5-carboxyspermylamide | transfer into mammalian primary | |
| | | endocrine cells with lipopolyamine- | |
| | | coated DNA. Proc. Natl. Acad. Sci. | |
| | | USA 86: 6982-6986; EPO Publication | |
| | | 0 394 111 | |
| DDAB | didoceyl methylammonium | | |
| | bromide | | |
| Dextran and dextran | DEAE-Dextran; Dextran sulfate | J Biol Chem. 2002. 277: 30208-30218. | |
| derivatives or | | Efficiency of protein transduction | |
| conjugates | | is cell type-dependent and is | |
| | | enhanced by dextran sulfate. | |
| | | Mai J C, Shen H, Watkins S C, | |
| | | Cheng T, Robbins P D. | |
| Diquaternary | (examples:) N,N′-dioleyl- | Bioconjug Chem 2001 Mar-Apr; 12(2): | Vical |
| ammonium salts | N,N,N′,N′-tetramethyl-1,2- | 258-63. Diquaternary ammonium | |
| | ethanediamine (TmedEce), N,N′- | compounds as transfection | |
| | dioleyl-N,N,N′,N′-tetramethyl-1,3- | agents. Rosenzweig H S, Rakhmanova V A, | |
| | propanediamine (PropEce), N,N′- | MacDonald R C.; U.S. Pat. No. 5,994,317 | |
| | dioleyl-N,N,N′,N′-tetramethyl-1,6- | | |
| | hexanediamine (HexEce), and their | | |
| | corresponding N,N′-dicetyl | | |
| | saturated analogues (TmedAce, | | |
| | PropAce and HexAce) | | |
| DLRIE | dilauryl oxypropyl-3-dimethylhydroxy | Ann N Y Acad Sci 1995 Nov 27; 772: | Vical |
| | ethylammonium bromide | 126-39. Improved cationic lipid | |
| | | formulations for in vivo gene | |
| | | therapy. Felgner P L, Tsai Y J, | |
| | | Sukhu L, Wheeler C J, Manthorpe M, | |
| | | Marshall J, Cheng S H. | |
| DMAP | 4-dimethylaminopyridine | | |
| DMPE | Dimyristoylphospatidylethanolamine | | |
| DMRIE | N-[1-(2,3-dimyristyloxy)propyl]- | Biochim Biophys Acta 1996 Jul 24; | |
| | N,N-dimethyl-N-(2-hydroxyethyl) | 1312(3): 186-96. Human | |
| | ammonium bromide | immunodeficiency virus type-1 (HIV-1) | |
| | | infection increases the sensitivity | |
| | | of macrophages and THP-1 cells to | |
| | | cytotoxicity by cationic liposomes. | |
| | | Konopka K, Pretzer E, Felgner P L, | |
| | | Duzgunes N. | |
| DMRIE-C | 1:1 formulation of N-[1-(2,3- | U.S. Pat. Nos. 5,459,127 and | Invitrogen (LTI) |
| | dimyristyloxy)propyl]-N,N- | 5,264,618, to Felgner, et al (Vical) | |
| | dimethyl-N-(2-hydroxyethyl) | | |
| | ammonium bromide (DMRIE) and | | |
| | cholesterol | | |
| DMRIE:DOPE | formulation of 1,2- | Hum Gene Ther 1993 Dec; 4(6): 781-8. | |
| | dimyristyloxypropyl-3-dimethyl- | Safety and short-term toxicity of a | |
| | hydroxyethyl ammonium bromide | novel cationic lipid formulation for | |
| | and dioleoyl phosphatidyl- | human gene therapy. San H, Yang | |
| | ethanolamine (DOPE) | Z Y, Pompili V J, Jaffe M L, Plautz | |
| | | G E, Xu L, Felgner J H, Wheeler C J, | |
| | | Felgner P L, Gao X, et al. | |
| DOEPC | dioleoylethylphosphocholine | | |
| DOHME | N-[1-(2,3-dioleoyloxy)propyl]-N- | | |
| | [1-(2-hydroxyethyl)]-N,N- | | |
| | dimethylammonium iodide | | |
| DOPC | dioleoylphosphatidylcholine | | |
| DOPC:DOPS | 1:1 (wt %) formulation of DOPC | | Avanti |
| | (dioleoylphosphatidylcholine) and | | |
| | DOPS | | |
| DOSPA | 2,3-dioleoyloxy-N-[2- | | |
| | (sperminecarboxamidoethyl]-N,N- | | |
| | di-met- hyl-1-propanaminium | | |
| | trifluoroacetate | | |
| DOSPA:DOPE | Formulation of 2,3-dioleoyloxy-N- | 3 Gene Med 2001 Jan-Feb; 3(1): 82-90. | |
| | [2-(sperminecarboxamidoethyl]- | Cationic liposome-mediated gene | |
| | N,N-dimethyl-1-propanaminium | transfer to rat salivary epithelial | |
| | trifluoroacetate (DOSPA) and | cells in vitro and in vivo. | |
| | dioleoyl phosphatidyl-ethanolamine | Baccaglini L, Shamsul Hoque A T, | |
| | (DOPE) | Wellner R B, Goldsmith C M, | |
| | | Redman R S, Sankar V, Kingman A, | |
| | | Barnhart K M, Wheeler C J, Baum B J. | |
| DOSPER | 1,3-Di-Oleoyloxy-2-(6-Carboxy- | Buchberger et al., 1996. DOSPER | Roche |
| | spermyl)-propylamid | liposomal transfection reagent: a | |
| | | reagent with unique transfection | |
| | | properties. Biochemica 2: 7-10. | |
| DOTAP | N-[1-(2,3-dioleoyloxy)propyl]- | | |
| | N,N,N-trimethyl-ammonium | | |
| | methylsulfate | | |
| DOTMA | N-[1-(2,3-dioleyloxy)propyl]-n,n,n- | | |
| | trimethylammoniumchloride | | |
| DPEPC | Dipalmitoylethylphosphatidyl- | | |
| | choline | | |
| Effectene | (non-liposomal lipid formulation | Histochem Cell Biol 2001 Jan; 115(1): | Qiagen |
| | used in conjunction with a special | 41-7. Long-term expression of | |
| | DNA-condensing enhancer and | foreign genes in normal human | |
| | optimized buffer) | epidermal keratinocytes after | |
| | | transfection with lipid/DNA complexes. | |
| | | Zellmer S, Gaunitz F, Salvetter J, | |
| | | Surovoy A, Reissig D, Gebhardt R. | |
| ExGen 500 | Apyrogenic solution of linear | Ferrari S., Moro E., Pettenazzo A., | Fermetas |
| | 22 kDa polyethylenimine (PEI) in | Behr J. P., Zacchello F., Scarpa M., | |
| | water | ExGen 500 is an efficient vector for | |
| | | gene delivery to lung epithelial | |
| | | cells in vitro and in vivo, Gene | |
| | | Ther, Oct; 4(10): 1100-1106, 1997 | |
| FuGENE 6 | (proprietary formulation) | J Neurosci Methods 1999 Oct 15; | Roche |
| | | 92(1-2): 145-52. Improved lipid- | |
| | | mediated gene transfer in C6 glioma | |
| | | cells and primary glial cells using | |
| | | FuGene. Wiesenhofer B, Kaufmann | |
| | | W A, Humpel C. | |
| GAP-DLRIE:DOPE | N-(3-aminopropyl)-N,N-dimethyl- | Hum Gene Ther 1996 Oct 1; 7(15): | |
| | 2,3-bis(dodecyloxy)-1- | 1803-12. A new cationic liposome DNA | |
| | propaniminium bromide/dioleyl | complex enhances the efficiency of | |
| | phosphatidylethanolamine | arterial gene transfer in vivo. Stephan | |
| | | D J, Yang Z Y, San H, Simari R D, | |
| | | Wheeler C J, Felgner P L, Gordon D, | |
| | | Nabel G J, Nabel E G | |
| GeneJammer | Proprietary polyamine | | Wako, USA |
| GeneJuice | Proprietary polyamine | | Novagen |
| GeneLimo | Proprietary liposomal formulations | | CPG, Inc. |
| | of polycationic lipids and a neutral, | | |
| | non-transfecting lipid compound | | |
| GeneSHUTTLE ™ | Novel extruded DOTAP and | | |
| | cholesterol (DOTAP:Chol) formulation | | |
| Genetransfer | Liposome-mediated | | Strategene |
| Genetransfer | | | Wako Pure Chemical |
| | | | (Japan) |
| GS 2888 cytofectin | | Proc Natl Acad Sci USA 1996 Apr 16; | Gilead Sciences |
| | | 93(8): 3176-81. A serum-resistant | |
| | | cytofectin for cellular delivery | |
| | | of antisense oligodeoxynucleotides | |
| | | and plasmid DNA. Lewis J G, Lin K Y, | |
| | | Kothavale A, Flanagan W M, Matteucci | |
| | | M D, DePrince R B, Mook R A Jr, | |
| | | Hendren R W, Wagner R W. | |
| Lipofectin ® | 1:1 (w/w) formulation of N-(1-2,3- | U.S. Pat. Nos. 4,897,355; 5,208,066; | Invitrogen (LTI) |
| | dioleyloxypropyl)-N,N,N- | and 5,550,289. | |
| | triethylammonium (DOTMA) and | | |
| | dioleylphosphatidylethanolamine | | |
| | (DOPE) | | |
| LipofectACE ™ | 1:2.5 (w/w) formulation of dimethyl | | Invitrogen (LTI) |
| | dioctadecylammonium bromide | | |
| | (DDAB) and dioleoyl | | |
| | phosphatidylethanolamine (DOPE) | | |
| LipofectAMINE ™ | 3:1 (w/w) formulation of 2,3- | U.S. Pat. No. 5,334,761; and | Invitrogen (LTI) |
| | dioleyloxy-N- | U.S. Pat. Nos. 5,459,127 | |
| | [2(sperminecarboxamido)ethyl]- | and 5,264,618, to Felgner, | |
| | N,N-dimethyl-1-propanaminium | et al. (Vical) | |
| | trifluoroacetate (DOSPA) and | | |
| | dioleoyl phosphatidylethanolamine | | |
| | (DOPE) | | |
| LipofectAMINE ™ | (proprietary formulation) | | Invitrogen (LTI) |
| 2000 | | | |
| LipofectAMINE | PLUS (proprietary formulation) and | U.S. Pat. Nos. 5,736,392 and 6,051,429 | Invitrogen (LTI) |
| PLUS ™ | LipofectAMINE ™ | | |
| LipoTAXI ® | (proprietary formulation) | Madry H, Trippel S B. Efficient | Stratagene |
| | | lipid-mediated gene transfer to | |
| | | articular chondrocytes. Gene Ther. | |
| | | 2000 Feb; 7(4): 286-91. | |
| monocationic | (examples:) 1-deoxy-1- | J Med Chem 2001 Nov 22; 44(24): | |
| transfection lipids | [dihexadecyl(methyl)ammonio]-D- | 4176-85. Design, synthesis, and | |
| | xylitol; 1-deoxy-1- | transfection biology of novel | |
| | [methyl(ditetradecyl)ammonio]-D- | cationic glycolipids for use in | |
| | arabinitol; 1-deoxy-1- | liposomal gene delivery. Banerjee R, | |
| | [dihexadecyl(methyl)ammonio]-D- | Mahidhar Y V, Chaudhuri A, Gopal V, | |
| | arabinitol; 1-deoxy-1- | Rao N M. | |
| | [methyl(dioctadecyl)ammonio]-D- | | |
| | arabinitol | | |
| O-Chol | 3 beta[1-ornithinamide-carbamoyl] | Gene Ther 2002 Jul; 9(13): 859-66. | |
| | cholesterol | Intraperitoneal gene delivery | |
| | | mediated by a novel cationic liposome | |
| | | in a peritoneal disseminated ovarian | |
| | | cancer model. Lee M J, Cho S S, | |
| | | You J R, Lee Y, Kang B D, Choi | |
| | | J S, Park J W, Suh Y L, Kim J A, | |
| | | Kim D K, Park J S. | |
| OliogfectAMINE ™ | (proprietary formulation) | | Invitrogen (LTI) |
| Piperazine based | Piperazine based amphilic cationic | U.S. Pat. Nos. 5,861,397 and 6,022,874 | Vical |
| amphilic cationic | lipids | | |
| lipids | | | |
| PolyFect | (activated-dendrimer molecules | | Qiagen |
| | with a defined spherical | | |
| | architecture) | | |
| Protamine | Protamine mixture prepared from, | Gene Ther 1997 Sep; 4(9): 961-8. | Sigma |
| | e.g., salmon, salt herring, etc.; can | Protamine sulfate enhances lipid- | |
| | be supplied as, e.g., a sulfate or | mediated gene transfer. Sorgi F L, | |
| | phosphate. | Bhattacharya S, Huang L. | |
| SuperFect | (activated-dendrimer molecules | Tang, M. X., Redemann, C. T., and | Qiagen |
| | with a defined spherical | Szoka, Jr., F. C. | |
| | architecture) | (1996) In vitro gene delivery by | |
| | | degraded polyamidoamine dendrimers. | |
| | | Bioconjugate Chem. 7: 703; published | |
| | | PCT applications WO 93/19768 | |
| | | and WO 95/02397 | |
| Tfx ™ | N,N,N′,N′-tetramethyl-N,N′-bis(2- | | Promega |
| | hydroxyethyl)-2,3-di(oleoyloxy)- | | |
| | 1,4-butanediammonium iodide] and DOPE | | |
| TransFAST ™ | N,N[bis(2-hydroxyethyl)-N- | | Promega |
| | methyl-N-[2,3-di(tetradecanoyloxy) | | |
| | propyl] ammonium iodide and DOPE | | |
| TransfectAce | | | Invitrogen (LTI) |
| TRANSFECTAM ™ | 5-carboxylspermylglycine | Behr et al. 1989. Proc. Natl. | Promega |
| | dioctadecylamide (DOGS) | Acad. Sci. USA 86: 6982-6986; | |
| | | EPO Publication 0 394 111 | |
| TransIT ®-LT1, | Proprietary combination of a | | Panvera, Minis |
| TransIT ®-LT2 and | nontoxic cellular protein & a | | |
| various other | proprietary polyamine | | |
| TransIT7 products | | | |
| TransMessenger | (lipid-based formulation that is used | | Qiagen |
| | in conjunction with a specific RNA- | | |
| | condensing enhancer and an | | |
| | optimized buffer; particularly useful | | |
| | for mRNA transfection) | | |
| Vectamidine | 3-tetradecylamino-N-tert-butyl-N′- | FEBS Lett 1997 Sep 8; 414(2): | |
| | tetradecylpropionamidine (a.k.a. | 187-92. The role of endosome | |
| | diC14-amidine) | destabilizing activity in the gene | |
| | | transfer process mediated by | |
| | | cationic lipids. El Ouahabi A, | |
| | | Thiry M, Pector V, Fuks R, | |
| | | Ruysschaert J M, Vandenbranden M. | |
| X-tremeGENE Q2 | (proprietary formulation) | | Roche Molecular |
| | | | Biochemicals |

What is claimed is:

1. A composition comprising a solid support with two or more features,
wherein each feature comprises viral particles which contain nucleic acid molecules and the nucleic acid molecules in different features differ in nucleotide sequence.

2. The composition of claim 1, wherein the viral particles are co-localized in the features on the solid support with a deposition agent.

3. The composition of claim 2, wherein the depositing agent contains at least one protein other than a protein normally associated with the viral particles.

4. The composition of claim 3, wherein the protein is an albumin, a histone, or a fibronectin.

5. The composition of claim 2, wherein the depositing agent contains at least one disaccharide.

6. The composition of claim 2, wherein the disaccharide is sucrose, maltose, or galactose.

7. The composition of claim 2, wherein the depositing agent contains at least one complex carbohydrate.

8. The composition of claim 7, wherein the complex carbohydrate is a gum, a pectin, or a carrageenan.

9. The composition of claim 8, wherein the gum is hydrocolloid gum.

10. The composition of claim 8, wherein the gum is selected from the group consisting of:
guar gum;
xanthan gum; and
locust bean gum.

11. The composition of claim 1, wherein the viral particles of at least one feature are co-localized on the solid support with a transfection reagent.

12. The composition of claim 1, wherein the nucleic acid molecules of at least one feature are either RNA molecules or DNA molecules.

13. The composition of claim 1, wherein the nucleic acid molecules of at least one feature encode a double-stranded RNA.

14. The composition of claim 1, wherein the double-stranded RNA is capable of knocking down gene expression by RNA interference.

15. The composition of claim 1, wherein the number of features on the solid support is in a range selected from the group consisting of:
2 to 10,000;
10 to 10,000;
100 to 10,000;
500 to 10,000;
1,000 to 10,000; and
2,000 to 10,000.

16. The composition of claim 1, wherein the density of the features is in a range selected from the group consisting of:
2 to 100 features per cm²;
5 to 300 features per cm²;
10 to 100 features per cm²;
10 to 2,000 features per cm²;
100 to 2,000 features per cm²
200 to 2,000 features per cm²;
400 to 2,000 features per cm²; and
800 to 2,000 features per cm².

17. The composition of claim 1, wherein the solid support is composed of glass or plastic.

18. The composition of claim 17, wherein the solid support is a glass microscope slide.

19. A composition comprising a solid support and two or more features, wherein each feature contains one or more carbohydrate.

20. A method of introducing a target biopolymer into a cell, the method comprising contacting the cell with a solid support which contains two or more features,
wherein the cell is contacted with a location on the solid support which contains either (a) the target biopolymer and a depositing agent containing at least one gum or (b) a viral particle which contains the target biopolymer.

21. The method of claim 20, wherein the target biopolymer is a nucleic acid.

22. The method of claim 21, wherein the nucleic acid is an expression vector.

23. The method of claim 22, wherein the expression vector encodes a double-stranded RNA.

24. The method of claim 23, wherein the encoded double-stranded RNA contains a double-stranded region which is between 20 and 30 nucleotides in length.

25. The method of claim 20, wherein the nucleic acid is a double-stranded RNA.

26. The method of claim 25, wherein the double-stranded RNA is between 20 and 30 nucleotides in length.

27. A method of making a compositions comprising a solid support with two or more features, the method comprising depositing viral particles on the solid support to produce the two or more features.

28. A method of making a compositions comprising a solid support with two or more features, the method comprising depositing a mixture of one or more target biopolymer and a depositing agent containing one or more gum on the solid support to produce the two or more features.

29. An array of target biopolymers comprising two or more dried spots on a solid support, wherein at least two of the dried spots comprise at least one target biopolymer and a depositing agent containing one or more gum.

30. The array of claim 29, wherein at least two of the dried spots each further comprise a transfection reagent.

31. A method of screening for at least one activity of a target biopolymer, the method comprising:
contacting a population of cells with an array comprising the two or more features which each contain different biopolymers; and
evaluating the contacted cells to detect a direct or an indirect activity associated with the introduction of at least one of the target biopolymers into the contacted cells.

32. A kit comprising a composition selected from the group consisting of:
a solid support with two or more features, wherein each feature comprises viral particles which contain nucleic acid molecules and the nucleic acid molecules in different features differ in nucleotide sequence;
a solid support and two or more features, wherein each feature contains one or more carbohydrate; and
an array of target biopolymers comprising two or more dried spots on a solid support,
wherein at least two of the dried spots comprise at least one target biopolymer and a depositing agent containing one or more gum.

33. The kit of claim 32, which further comprises one or more component selected from the group consisting of:
one or more cell line;
one or more buffer;
one or more enzyme;
one or more transfection reagent;
one or more culture medium or culture medium component; and
one or more set of instructions for using one or more kit component.

34. A method for supplying a product to a customer, the method comprising:
taking an order for the product; and
supplying the product to the customer,
wherein the product is a kit of claim 32.

35. The method of claim 34, wherein the customer is also sent a bill for the product.

36. A method for advertising a product, the method comprising:
preparing an advertisement for the product; and
publicly disclosing the advertisement,
wherein the product is a kit of claim 32.

37. The method of claim 36, wherein the advertisement is selected from the group consisting of:
a magazine advertisement;
a flyer or brochure for mailing to customers or potential customers; and
a web page or web accessible document.
